(19) United States
(12) Patent Application Publication
(10) Pub. No.: US 2004/0158194 A1
(43) Pub. Date: Aug. 12, 2004

(54) Oral devices and methods for controlled drug release

(75) Inventors: Andy Wolff, Harutzim (IL); Ben Z. Beiski, Kiryat Ono (IL); Yoram Sela, Ra'anana (IL)

Correspondence Address: G.E. EHRLICH (1995) LTD., c/o ANTHONY CASTORINA, SUITE 207, 2001 JEFERSON DAVIS HIGHWAY, ARLINGTON, VA, 22202, US

(73) Assignee: WOLFF Andy and BEISKI Ben Z.

(21) Appl. No.: 10/668,274

(22) Filed: Sep. 24, 2003

Related U.S. Application Data
(60) Provisional application No. 60/445,228, filed on Feb. 6, 2003.

(51) Int. Cl. A61M 31/00

(52) U.S. Cl. 604/66

(57) ABSTRACT

Drug dosage forms, which are housed in oral devices, and methods for controlled drug release are provided. The oral devices are permanently or removably inserted in the oral cavity and refilled or replaced as needed. The controlled drug release may be passive, based on the dosage form, or electronically controlled, for a high-precision, intelligent, drug delivery. Additionally, the controlled release may be any one of the following: release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system. Drug absorption in the oral cavity may be assisted by an electrotransport mechanism. The oral devices require refilling or replacement at relatively long intervals of weeks or months, maintain a desired dosage level in the oral cavity, hence in the gastrointestinal tract, for extended periods, address situations of narrow drug therapeutic indices, and by being automatic, ensure adherence to a prescribed medication regimen.

This Application claims priority from U.S. Provisional Application No. 60/445,228, filed Feb. 6, 2003.

### FIELD AND BACKGROUND OF THE INVENTION

The present invention relates to controlled drug release, and more particularly, to oral devices and methods that provide various drug release schedules.

Oral drug administration is the most common drug delivery route; some 55% of the drug market are targeted for that route. It would be desired for the drug to be delivered at a controlled rate from the gastrointestinal tract, to maintain a controlled level of the drug in the blood stream and the tissue, and to control diurnal variations, resulting from oral intake at specific times during the day, by the patient. Yet, bioavailability of orally administered drugs, the degree to which the drug is available to the target tissue, is affected by drug dissolution, drug degradation in the gastrointestinal tract, and drug absorption, and is generally not constant with time. Some drugs have high bioavailability and may be dissolved and absorbed too fast, so as to peak shortly after intake. In these cases, controlled release dosage forms attempt to slow down the dissolution process. Others have very low bioavailability and may be eliminated by the gastrointestinal tract before they are absorbed. In these cases, approaches that increase absorption and approaches that increase gastrointestinal retention may be employed.

The absorption of a drug (or of a drug precursor) into the systemic circulation is determined by the physicochemical properties of the drug, its formulations, and the route of administration, whether oral, rectal, topical, by inhalation, or by intravenous administration. Oral administration includes swallowing, chewing, sucking, as well as buccal administration, i.e., placing a drug between the gums and cheek, and sublingual administration, i.e., placing a drug under the tongue. The advantage of chewing, sucking, as well as buccal and sublingual administration is that they lead also to direct absorption via the oral cavity, a route that avoids both the gastrointestinal tract and its losses, and the pre-systematic, first-pass metabolism, in the liver. A prerequisite to absorption is drug dissolution.

The extent of drug dissolution depends on whether the drug is in salt, crystal, or hydrate form. To improve dissolution, disintegrants and other excipients, such as diluents, lubricants, surfactants (substances which increase the dissolution rate by increasing the wetability, solubility, and dispersibility of the drug), binders, or dispersants are often added during manufacture.

Drug degradation in the gastrointestinal tract is due to the numerous gastrointestinal secretions, low pH values, and degrading enzymes. Since luminal pH varies along the gastrointestinal tract, the drug must withstand different pH values. Interaction with blood, food staff, mucus, and bile may also affect the drug. Reactions that may affect the drug, and reduce bioavailability are complex is formations, for example, between tetracycline and polyvalent metal ions, hydrolysis by gastric acid or digestive enzymes, for example, penicillin and chloramphenicol palmitate hydrolysis, conjugation in the gut wall, for example, sulfoconjugation of isoproterenol, adsorption to other drugs, for example, digoxin and cholestyramine, and metabolism by luminal microflora.

Overall, low bioavailability is most common with oral dosage forms of poorly water-soluble, slowly absorbed drugs. Insufficient time in the gastrointestinal tract is another common cause of low bioavailability. Ingested drug is exposed to the entire gastrointestinal tract for no more than 1 to 2 days and to the small intestine for only 2 to 4 hours. If the drug does not dissolve readily or cannot penetrate the epithelial membrane quickly, its bioavailability will be low. Age, sex, activity, genetic phenotype, stress, disease (e.g., achlorhydria, malabsorption syndromes), or previous GI surgery can further affect drug bioavailability.

Table 1 below [Encyclopedia of Controlled Drug Delivery, volume 2, edited by Edith Mathiowitz] summarizes some parameters of the oral route that affect drug bioavialablity.

TABLE 1

| | | LIQUID | | TRANSIT |
|---|---|---|---|---|
| | AREA, | SECRETION, | PH | TIME, |
| SECTION | M² | LITER/DAY | VALUE | HOUR |
| Oral cavity | ˜0.05 | 0.5-2 | 5.2-6.8 | Short |
| Stomach | 0.1-0.2 | 2-4 | 1.2-3.5 | 1-2 |
| Duodenum | ˜0.04 | 1-2 | 4.6-6.0 | 1-2 |
| Small | 4500 | 0.2 | 4.7-6.5 | 1-10 |
| Intestine | (including | | | |
| | microvillies) | | | |
| Large | 0.5-1 | ˜0.2 | 7.5-8.0 | 4-20 |
| Intestine | | | | |

In addition to the physical barrier of the epithelial cells, chemical and enzymatic barriers affect drug absorption.

Another important barrier to drug absorption is the pre-systematic, first-pass metabolism, primarily hepatic metabolism. The predominant enzymes in this metabolism are the multi-gene families of cytochrome P450, which have a central role in metabolizing drugs. It appears that variations in P450s between individuals lead to variations in their ability to metabolize the same drug.

Additionally, multidrug resistance (MDR) may be a barrier to drug absorption. MDR, which is a major cause of cancer treatment failure, is a phenomenon whereby cancer cells develop a broad resistance to a wide variety of chemotherapeutic drugs. MDR has been associated with overexpression of P-glycoprotein (P-gp) or multidrug resistance-associated protein (MRP), two transmembrane transporter molecules which act as pumps to remove toxic drugs from tumor cells. P-glycoprotein acts as a unidirectional efflux pump in the membrane of AML cells and lowers the intracellular concentration of cytotoxic agents, by pumping them out of leukemic cells. Yet it confers resistance to a variety of chemotherapy drugs, including daunorubicin.

Approaches for Increased Drug Absorption:

Except for the route of intravenous administration, after dissolution, a drug must traverse several semi permeable biologic barriers before reaching the systemic circulation. A drug may cross the biologic barrier by passive diffusion, or by other naturally occurring transfer modes, for example, facilitated passive diffusion, active transport, or pinocytosis. Alternatively, a drug may be artificially assisted to cross the biologic barrier.

In passive diffusion, transport depends on the concentration gradient of the solute across the biologic barriers. Since the drug molecules are rapidly removed by the systemic circulation, drug concentration in the blood is low compared with that at the administration site, producing a large concentration gradient. The drug diffusion rate is directly proportional to that gradient. Yet, the drug diffusion rate also depends on other parameters, for example, the molecule's lipid solubility and size. Because cell membranes are lipoid, lipid-soluble drugs diffuse more rapidly through cell membranes than relatively lipid-insoluble drugs. Additionally, small drug molecules penetrate biologic barriers more rapidly than large ones.

Another naturally occurring transfer mode is facilitated passive diffusion, which occurs for certain molecules, such as glucose. It is believed that a carrier component combines reversibly with a substrate molecule at the cell membrane exterior. The carrier-substrate complex diffuses rapidly across the membrane, releasing the substrate at the interior surface. This process is characterized by selectivity and saturability: The carrier is operative only for substrates with a relatively specific molecular configuration, and the process is limited by the availability of carriers.

An alternative is nanotechnology, which derives its name from the size of the objects that it deals with. These are objects that are usually smaller than 100 nanometers, and may be at the molecular scale. As related to pharmaceuticals, the drugs particle are reduce to “nano” size, for smoother release, better dissolution pattern, better control on absorption, and decreasing the required dose.

Active transport, which is another naturally occurring transfer mode, appears to be limited to drugs that are structurally similar to endogenous substances. Active transport is characterized by selectivity and saturability and requires energy expenditure by the cell. It has been identified for various ions, vitamins, sugars, and amino acids.

Still another naturally occurring transfer mode is pinocytosis, in which fluids or particles are engulfed by a cell. The cell membrane encloses the fluid or particles, then fuses again, forming a vesicle that later detaches and moves to the cell interior. Like active transport, this mechanism requires energy expenditure. It is known to play a role in drug transport of protein drugs.

The foregoing discussion relates to naturally occurring transfer modes. Where these are insufficient, for example, in cases of macromolecules and polar compounds, which cannot effectively traverse the biological barrier, drug transport may be artificially induced.

Electrotransport refers generally to electrically induced passage of a drug (or a drug precursor) through a biological barrier. Several electrotransport mechanisms are known, as follows:

Iontophoresis involves the electrically induced transport of charged ions, by the application of low level, direct current (DC) to a solution of the medication. Since like electrical charges repel, the application of a positive current drives positively charged drug molecules away from the electrode and into the tissues; similarly, a negative current will drive negatively charge ions into the tissues. Iontophoresis is an effective and rapid method of delivering water-soluble, ionized medication. Where the drug molecule itself is not water-soluble, it may be coated with a coating, for example, sodium lauryl sulfate (SLS), that may form, water soluble entities.

Electroosmosis involves the movement of a solvent with the agent through a membrane under the influence of an electric field.

Electrophoresis is based on migration of charged species in an electromagnetic field. Ions, molecules, and particles with charge carry current in solutions when an electromagnetic field is imposed. Movement of a charged species tends to be toward the electrode of opposite charge. The voltages for continuous electrophoresis are rather high (several hundred volts).

Electroporation is the process in which a biological barrier is subjected to a high voltage alternating-current (AC) surge, or pulse. The AC pulse creates temporary pores in the biological membrane, specifically between cells. The pores allow large molecules, such as proteins, DNA, RNA, and plasmids to pass through the biological barrier.

Iontophoresis, electroosmosis, and electrophoresis are diffusion processes, in which diffusion is enhanced by electrical or electromagnetic driving forces. In contrast, electroporation literally punctures the biological barriers, along cell boundaries, enabling passage of large molecules, through.

Generally a combination of more than one of these processes is at work, together with passive diffusion and other naturally occurring transfer modes. Therefore, electrotransport refers to at least one, and possibly a combination of the aforementioned transport mechanisms, which supplement the naturally occurring transfer modes.

Medical devices that include drug delivery by electrotransport are described, for example, in U.S. Pat. No. 5,674,196, to Donaldson, et al., U.S. Pat. No. 5,961,482, to Chien, et al., U.S. Pat. No. 5,983,131, to Weaver, et al., U.S. Pat. No. 5,983,134, to Ostrow, and U.S. Pat. No. 6,477,410, to Henley, et al., all of whose disclosures are incorporated herein by reference.

In addition to the aforementioned electrotransport processes, there are other electrically assisted drug delivery mechanisms, as follows:

Sonophoresis, or the application of ultrasound, induces growth and oscillations of air pockets, a phenomenon known as cavitation. These disorganize lipid bilayers thereby enhancing transport. For effective drug transport, a low frequency of between 20 kHz and less than 1 MHz, rather than the therapeutic frequency, should be used. Sonophoresis devices are described, for example, in U.S. Pat. Nos. 6,002,961, 6,018,678, and 6,002,961 to Mitragotri, et al., U.S. Pat. Nos. 6,190,315 and 6,041,253 to Kost, et al. U.S. Pat. No. 5,947,921 to Johnson, et al. and U.S. Pat. Nos. 6,491,657, and 6,234,990 to Rowe, et al., all of whose disclosures are incorporated herein by reference.

Ablation, or the literal slicing of tissue, by various means, is another method of forcing drugs through a biological barrier. In addition to mechanical ablation, for example with hyperdemic needles, one may use laser ablation, cryogenic ablation, thermal ablation, microwave ablation, radiofrequency ablation or electrical ablation. In essence, electrical ablation is similar to electroporation, but tends to be more severe.

U.S. Pat. No. 6,471,696, to Berube, et al., describes a microwave ablation catheter, which may be used as a drug delivery device. U.S. Pat. No. 6,443,945, to Marchitto, et al., describes a device for pharmaceutical delivery using laser ablation. U.S. Pat. No. 4,869,248, to Narula describes a catheter for performing localized thermal ablation, for purposes of drug administration. U.S. Pat. Nos. 6,148,232 and 5,983,135, to Avrahami, describe drug delivery systems by electrical ablation. The disclosures of all of these are incorporated herein by reference.

Controlled Release Dosage Forms:

Oral controlled-release dosage forms are often designed to maintain therapeutic drug concentrations for at least 12 hours. Several controlled release mechanisms may be used, for example, as taught by Encyclopedia of Controlled Drug Delivery, volume 2, edited by Edith Mathiowitz, pp. 838-841. These are based on the use of specific substances, generally polymers, as a matrix or as a coating. These may be materials that degrade fast or slowly, depending on the desired effect. For example, when a drug's half-life in the body is too short, the drug may be coated with a slowly dissolving coating. Consequently, the drug must diffuse through the coating, and its half-life is slowed. Other coating materials form pores that fill with gastrointestinal fluids, increase the contact area between the drug and the gastrointestinal fluids, and reduce the diffusion path in the drug matrix, so as to increase the drug half-life. In these and other manners, modified drug release forms prolong, delay or sustain the release of a drug in a passive, controlled manner, wherein passive refers to systems not controlled by electronics. A summary of modified drug release forms, for passive, controlled release, is as follows:

Osmotic systems rely on the uptake of water by the dosage form to increase the osmotic pressure within the system. The build up of osmotic pressure drives the drug through an orifice in the dosage form to release the drug in a controlled manner.

Membrane-coated tablets consist of water-soluble drug particles compressed to form a tablet core. A coating of a substantially insoluble polymer, for example, polyvinyl chloride, is applied to the tablet core, wherein the coating is mixed with a water soluble, pore-forming compound. Additionally, the solubility of the pore-forming compound may be pH dependent, to target a specific zone in the gastrointestinal tract. The rate of drug release is dependent on the pH level and on the extent of porosity in the coating, after the pores are formed.

Enteric-coated dosage forms are dosage forms in which a drug core is coated with a polymeric mixture, formed of soluble and insoluble particles. The soluble particles dissolve in the intestinal fluids, exposing the insoluble particles. As a result, a micro porous layer is formed around the drug core and the drug slowly permeates through the pores.

Multilayered tablets consist of a drug core layered with several coatings, which may be of different solubility, to provide release at specific time intervals and (or) pH levels. As each layer dissolves, a pulsatile-type release is achieved. By modifying the types and amount of polymers use, the release rate can be adjusted.

pH independent controlled release tablets are produced by wet granulating an acidic or basic drug blend with a buffering agent and appropriate excipients. The granules are then coated with a film, which is permeable to gastrointestinal fluid, and the coated composite is compressed into a tablet. Upon oral administration, gastrointestinal fluid permeates the film coating. When in contact with the gastrointestinal fluid, the buffering agents adjust the pH value of the tablet; the drug dissolves and permeates out at a constant rate, independent of the pH level in the gastrointestinal tract.

A Hydrogel plug dosage form consists of a capsule having a water insoluble body sealed with a water-soluble cap, which further contains a hydrogel plug. When the capsule is swallowed, the water-soluble cap dissolves and exposes the hydrogel plug, which begins to swell. At a predetermined time after ingestion, the hydrogel plug is ejected and the drug is released into the gastrointestinal tract.

Multiparticulate dosage forms generally consist of sugar or nonpareil pellets, spray coated with a drug, dried, then spray coated with a second coating composition, which provides controlled release. The second coating composition is typically formed of polymers, which are partially soluble or insoluble in the gastric fluid, wherein the degree of solubility depends on the desired drug release pattern. The doubly coated pellets are placed in a capsule, for swallowing. A capsule can contain pellets of different types and release profiles.

Gastro-Retention Devices:

Many of the orally administered drugs are absorbed efficiently in the upper gastrointestinal tract, the stomach, and the proximal section of the small intestine but barely in the colon. [Singh at all. J Controlled Release 63 (3),235 (2000), and U.S. Pat. No. 5,443,843, to Curatolo at al.] Yet, because the passage of the drug in the upper gastrointestinal tract, the stomach, and the proximal section of the small intestine is relatively fast, generally about 12 hours, drug bioavailability is limited—a dosage form is operative primarily during that time span. Prolonging the retention time of the drug in the upper sections is of outmost importance for increased bioavailability. [Hwang at al. Crit. Rev. Ther. Drug Carrier Syst, 15(3),243 (1998).]

An answer may be a long-term gastric retention device, which is taken orally and which is adapted for long-term drug release in the upper gastrointestinal tract. A long-term gastric retention device may be especially useful in cases of drugs taken over long periods, as in instances of chronic diseases and hormonal treatments. It will also simplify treatments that combine different drugs.

The medication that may be considered for long-term gastric retention devices must fit the following criteria:

1. Large therapeutic range, so that deviations from the amount of released drug, above or below the predicted level, will not cause significant symptoms; and

2. Overdoses will not endanger the patient.

Potential drug candidates include: Analgesics, Anxiolytics, Antimigroine drugs, Sedatives, Antipsihotics, Anticonvulsants, Antiparcinsons, Antiallergic drugs, Antidepressants, Antiemetics, Astma-profilactics, Gastric-hypoacidics, Anticonstipation drugs, Intestinal antiinflammatory agents, Antihelmintics, Antianginals, Diuretics, Hypolipidemic agents, Anti-inflammatory drugs, Hormones, Vitamins, Antibiotics.

Several approaches for long-term gastric retention device are available, as follows:

1. An intragastric floating system: This system is designed to float in the gastric fluid. Three major techniques have been used to generate buoyancy in the gastric fluid, as follows:

i. A mixture of bicarbonate and gastric fluid generates CO₂, which remains trapped within a matrix of the dosage form, causing it to float in the stomach, so as to prolong its residence in the stomach. Similarly, another gas may be produced.

ii. A low-density core system is formed of buoyant materials, such as air, CO₂ or gels. It is coated by an outer layer of a dosage form, adapted for controlled release.

iii. A gel forming hydrophilic polymer, which upon contact with the gastric fluid forms a gelatinous shell, may be used to produce a hydrodynamic-balanced system, whose buoyancy is ensured by its dry or hydrophobic core. The gelatinous shell is responsible also for the controlled release of the drug.

Yet, these floating devices have a stomach residence time of only a few hours, and their action is dependent upon the amount of food and water in the stomach. Thus, their performance is nonuniform and difficult to predict.

2. High density system: This system is based on sinking the device to the bottom of the stomach. Thus, the device is usually made of heavy materials. Initially, this approach looked promising, but studies have since shown that there is no appreciable gastric retention.

3. A Mucoadhesive system: This adhesive system is able to adhere to the mucous walls of the stomach, and is expected to remain in the stomach, for the duration of the mucous layer turnover. Yet, it also binds to almost any other material it comes in contact with, gelatin capsules, proteins, and free mucous, in the gastric fluid. Another obstacle is that its adhesiveness is pH-dependent, and higher than normal gastric pH levels reduce the adhesiveness dramatically. Thus, experimental results were disappointing, and no substantial increase in residence time in the stomach was observed.

4. A Magnetic system: an extracorporeal magnet is placed over the stomach, and small magnetized particles, within the dosage form, prevent the it from leaving the stomach. Even through some success has been reported, the viability of these systems is in doubt, because of the need to carry the extracorporeal magnet, placed very accurately, in order to obtain the desired results. New, more convenient ways to apply a magnetic field have to be found to improve this concept.

5. An expansible system: This system is based on a sharp dimensional change, in the stomach. Several methods have been proposed:

i. a hydrogel that swells upon contact with the gastric fluid;

ii. an osmotic devise that contains salt or sugar, surrounded by a semi-permeable membrane;

iii. a system containing a low boiling liquid, that turns into gas at body temperature and inflates the device to its desired size, wherein simultaneous with the swelling, controlled release begins.

Yet, these systems suffer from a slow swelling rate and therefore are not retained in the stomach. Furthermore, the ability to swell to a desired size and the degradation process that follows still pose substantial challenges.

6. A superporous, biodegradable, hydrogel system: This system is based on the swelling of a unique hydrogel system, superporous hydrogel, synthesized by cross-linking polymerization of various vinyl monomers in the presence of gas bubbles formed by chemical reaction of acid and NaHCO₂. Compared to other expansible systems, it has a much higher swelling level and swells at a much faster rate than conventional hydrogels, attaining a desired expanded form in minutes, as opposed to hours. Yet, the system is mechanically weak, so it breaks down, leading to a short residence times in the stomach.

7. A mechanical, expansible system: This system is based on a mechanical device, which unfolds or extends from an initial, compact size, to an extended form that prevents passage through the gastric pylorus. At present, the mechanical expansible system is the most promising, in the gastric retention field, yet many technical problems, related to its performance are yet to be solved.

Thus, at present, reliable and efficient long-term gastric retention devices are not available.

Patient Adherence to Prescription Schedule.

Low adherence with prescribed treatments is ubiquitous, yet it may undermine the success of a treatment. Typical adherence rates are about 50% for medications and are much lower for lifestyle prescriptions and other more behaviorally demanding regimens. [Haynes R B, McDonald H P, Garg A X. JAMA 288(22):2880-3 (2002)]. In fact, a Hungarian study reported that one third of hypertension patients took the medication irregularly, despite the potentially life-threatening implications. [Rapi J. Orv Hetil 143(34):1979-83 (2002)] Another survey showed that 62.4% patients with familial hypercholesterolemia were not taking their prescribed cholesterol-lowering medication. [Umans-Eckenhausen M A, Defesche J C, van Dam M J, Kastelein J J. Arch Intern Med 163(1):65-8 (2003).] In fact, missed doses occurs more frequently than taking an overdose. [De Kierk E, Van Der Heijde D, Landewe R, Van Der Tempel H, Urquhart J, Van Der Linden S. J Rheumatol 30(1):44-54 (2003).]

Current methods of improving medication adherence for chronic health problems are complex, labor-intensive, and not very effective. Improving adherence to long-term regimens requires a combination of information about the regimen, counseling about the importance of adherence, advice on how to organize medication regimen in your life, reminders, rewards and recognition for the patient's efforts to follow the regimen, and social support from family and friends. The full benefit of medication is not realized at low levels of adherence; therefore, more studies and innovative approaches to assist patients to follow prescriptions are needed. [McDonald H P, Garg A X, Haynes R B. JAMA 288(22):2868-79 (2002).]

Another issue in drug prescription is the efficacy and safety of both new and existing drugs. Efficacy and safety are related factors in a drug's clinical profile. Drug doses are calculated according to a therapeutic window for each drug, which is the range of drug concentration in the blood, ranging between the minimum effective therapeutic concentration and the minimum toxic concentration. The width of the therapeutic window can be measured by a therapeutic index, which is the ratio between the median lethal dose and the median effective dose. This is a safety margin for using a specific drug. The wider the index, the safer the drug.

The accepted rule in pharmaceutics is that a drug that has less than a twofold difference between its toxic and effective doses is considered to have a “narrow therapeutic index,” and its use must be carefully monitored. Yet, several clinically important drugs have narrow therapeutic indices. These include anti-AIDS agents like AZT, antibiotics like ciprofloxacin, CNS agents like Levodopa, and anti diabetic agents.

Chronotherapy:

According to Stehlin [Stehlin I., “A Time to Heal: Chronotherapy Tunes In to Body's Rhythms,” US Food and Drug Administration, http://www.fda.gov/fdac/features/1997/397_chrono.html], our body's physiological clock takes its cue from the solar system, affecting blood pressure, blood coagulation, blood flow, and other functions. Several types of physiological cycles may be defined, as follows:

ultradian, which are cycles shorter than a day (for example, sleep cycles of about 90 minutes);

circadian, which are daily cycles (such as sleeping and waking patterns);

infradian, which are cycles longer than 24 hours (for example, monthly menstruation); and

seasonal (for example, a seasonal affective disorder (SAD), which causes depression in susceptible people during the short days of winter).

For example, the normal lung function undergoes circadian changes and reaches a low point in the early morning hours. This dip is particularly pronounced in people with asthma.

Thus, chronotherapy may be especially useful for asthma. It is aimed at getting maximal effect from bronchodilator medications during the early morning hours. For example, the bronchodilator Uniphyl, a long-acting theophylline preparation, manufactured by Purdue Frederick Co. of Norwalk, Conn., and approved by FDA in 1989 may be used for chronotherapy. Taken once a day in the evening, Uniphyl causes theophylline blood levels to reach their peak and improve lung function during the early morning hours.

Additionally, according to Stehlin, chronotherapy may be useful in the treatment of cancer, arthritis, hypertension, diabetes, hear attacks, sexual dysfunction, and eating and sleeping disorders. For example, animal studies suggest that chemotherapy may be more effective and less toxic if cancer drugs are administered at carefully selected times. It appears that there may be different chronobiological cycles for normal cells and tumor cells. Thus, if administration of cancer drugs is timed with the chronobiological cycles of tumor cells, it will be more effective against the cancer and less toxic to normal tissues.

Furthermore, chronobiological patterns have been observed with arthritis pain. People suffering from osteoarthritis, the most common form of the disease, tend to be in pain at night. But for people with rheumatoid arthritis, the pain usually peaks in the morning. When using chronotherapy for arthritis, both nonsteroidal anti-inflammatory drugs and corticosteroids may be timed to ensure that the highest blood levels of the drug coincide with the times of peak pain.

Dental Structure and Dental Implements:

The following is a brief overview of a tooth structure and of known techniques of dental repair and reconstruction, which relate to the present invention. FIG. 1 is a cross-sectional view of a tooth 10, as taught, for example, by http://www.dentalreview.com/tooth_anatomy.htm As seen in the figure, the basic parts of a tooth are: a crown 12, the portion of tooth above a gum 14, and a root or roots 16, which anchor the tooth in a jawbone 15. A pulp 18 is arranged within a pulp chamber 20 and within a root canal or root canals 22.

Crown 12 is formed of an inner structure of dentine 26 and an external layer of enamel 24, which defines a chewing surface 28. There may be one, two, or more roots 16. Each has an external layer of cement 30, inner structure of dentine 26, and one root canal 22. Pulp 18 is formed of tiny blood vessels, which carry nutrients to the tooth, and nerves, which give feeling to the tooth. These enter root canals 22 via accessory canals 32 and root-end openings 34.

Tooth 10 may define a cylindrical coordinate system of a longitudinal axis x, and a radius r. A coronal or incisal end 36 may be defined as the end above gum 14 and a apical end 38 may be defined as the end below it.

Various intraoral devices and dental reconstruction and repair methods that relate to the present invention are reviewed in conjunction with FIGS. 2A-7C, hereinbelow.

Root Canal:

A root canal treatment may be required when the pulp is diseased or injured and dies. Common causes of pulp death are a deep cavity, a cracked filling, or a cracked tooth. Bacteria then invade the tooth and infect the pulp. The inflammation and infection may spread down the root canal, often causing sensitivity to hot or cold foods and pain.

Root canal treatment involves removing the diseased pulp and cleaning and sealing the pulp chamber and root canals, then filling or restoring the crown. The steps in root canal therapy are described, for example, in http://your-doctor.com/patient_info/dental_info/dental_disorders/rootcanal.html#1, “Root Canal (Endodontic) Therapy,” and are illustrated in FIGS. 2A-2G below.

FIGS. 2A-2C illustrate a root canal treatment in which crown 12 was not severely damaged. As seen in FIG. 2A, an opening 40 is made, generally through crown 12 and dentine 26, into pulp chamber 20. Pulp 18 (FIG. 1) is then removed with a tiny file (not shown), and pulp chamber 20 and root canals 22 are cleaned and shaped to a form that can be filled.

As seen in FIG. 2B, medications 42 may be applied to pulp chamber 20, and root canals 22, for a period of about two weeks, to disinfect them. A temporary filling 44 may be placed in crown opening 40 to protect the tooth between dental visits.

As seen in FIG. 2C, after removing medications 42 and temporary filling 44 of FIG. 2B, pulp chamber 20 and root canals 22 are cleaned and filled with a permanent filling 46, and chewing surface 28 is restored.

FIGS. 2D-2G illustrate situations in which crown 12 (FIG. 1) was severely damaged. As seen in FIG. 2D, remnants of crown 12 are removed, and root canals 22 are cleaned and shaped as above.

As seen in FIG. 2E, medications 42 may be applied to root canals 22, for a period of about two weeks, to disinfect them. A sealing layer 27 may then be applied over the exposed dentine, to protect it until the next dental visit.

As seen in FIG. 2F, after removing medications 42 of FIG. 2E, root canals 22 are cleaned and filled with permanent filling 46. A core 29 of permanent filling 46 is then constructed over the roots, to restore the crown, and a mold (not shown) is taken of the remaining tooth structure and core 29. A temporary structure 50 is then placed over the remaining tooth structure and core 29.

As seen in FIG. 2G, a permanent, enamel-like structure 52 is prepared from the mold, and placed over core 29.

On the other hand, when teeth are lost, replacement options include bridges implant and dentures.

Bridge:

A bridge may be used to fill a gap of up to four teeth, where there are healthy natural teeth on either side of the gap. FIGS. 3A-3F illustrate an application of a three-unit bridge 60 between two healthy teeth 62 and 64.

As seen in FIGS. 3A-3B, the dentist will prepare teeth 62 and 64 on either side of the gap by removing portions of the enamel and dentin, leaving stumps 66 and 68. Impressions or molds of stumps 66 and 68 and the gap between them are taken for the construction of the bridge. In the meantime, a temporary bridge is applied to protect the exposed stumps and provisionally restore the missing teeth.

As seen in FIGS. 3C-3D, the dentist then fits bridge 60, which includes a prosthetic tooth crown 70, over stumps 66 and 68. If the fit is good, he cements bridge 60 into place, restoring function to the area.

FIGS. 3E-3F illustrate an alternative technique: a bridge 72 may be formed of prosthetic tooth crowns 70 and anchors 74, adapted to clamp onto healthy teeth 62 and 64. Unlike bridge 60 of FIGS. 3C-3D, which is cemented into place, bridge 72 may be removed, for example, for cleaning.

Dental Implant:

As an alternative to a bridge, a dental-implant-and-prosthetic-tooth-crown 80 may be used. As seen in FIGS. 4A-4C, dental-implant-and-prosthetic-tooth-crown 80 includes, for example, a dental implant or fixture 82, surgically implanted into the bone, which grows around it. Once dental implant 82 is is anchored in the bone, a stump 84 is mounted on it and prepared to accept prosthetic tooth crown 70.

Dentures:

When several teeth are missing, dentures 90 can be used, containing a plurality of prosthetic tooth crowns 70, as seen in FIGS. 5A-5C.

It is possible to get either full dentures, of all the teeth, as seen in FIG. 5A, or partial dentures, of fewer teeth, as seen in FIG. 5B. Full dentures are form-fitted to the gum ridges, creating an adhesive effect that keeps them in place. Partial dentures may be adapted to fit around the natural teeth, to help them stay in place. Additionally, as seen in FIG. 5C, a dental implant post 82 may be used to further to secure the dentures.

Crown:

At times, the root of the tooth is intact. But its upper portion is severely decayed or broken. An artificial crown may then be placed on the tooth, as seen in FIGS. 6A-6C.

FIG. 6A illustrates a broken tooth 92. As seen in FIG. 6B, it is prepared by removing a portion of the enamel and dentin, exposing a stump 94. As seen in FIG. 6C, a crown 96 is then cemented over stump 94, restoring the chewing surface.

Braces:

Other known dental devices include braces for orthodontics. FIG. 7A illustrates braces 100, which include molar bands 102, arch wires 104, and brackets 106.

Alternatively, FIG. 7B illustrates braces 110, which includes a metal or plastic plate 112, adapted to fit against the roof of the mouth, and wires 114 and 116. Alternatively, FIG. 7C illustrates invisible braces 120. In general, the braces of FIGS. 7A-7C may be easily removed, for example, for cleaning.

Slow-releasing devices to be attached to or placed around teeth or implanted into the gum are disclosed, for example, in U.S. Pat. Nos. 3,624,909; 3,688,406; 4,020,558; 4,175,326; 4,681,544, 4,685,883, 4,837,030 and 4,919,939. These devices deliver a medication into the oral cavity, but they lack a controlled rate of delivery for extended time periods which is of utmost importance in the prevention and treatment of the heretofore mentioned diseases and conditions. For example, U.S. Pat. No. 4,837,030 discloses an orally administrable pharmaceutical composition comprising is beads coated with an ultra-thin layer of a polymer that erodes under gastric conditions. When suspended in water, more than 90% of the pharmaceutical agent is released from the composition between 20 to 90 minutes; U.S. Pat. No. 4,919,939 discloses a controlled release drug delivery system comprising a polymeric matrix, which dissolves, releasing the drug contained therein within 10 to 18 hours, upon the action of the saliva.

U.S. Pat. No. 5,614,223, to Sipos, entitled, “Intraoral medicament-releasing device,” describes controlled rate-release devices for releasing a pharmaceutically active agent into the oral cavity by the dissolving action of the saliva, a process of preparing such devices and methods of preventing/treating conditions/diseases in a mammal by delivering a pharmaceutically active substance into the oral cavity.

U.S. Pat. No. 5,686,094, to Acharya, entitled, “Controlled release formulations for the treatment of xerostomia,” describes controlled or sustained dosage forms, and in particular certain polymeric matrices or complexes which are suitable for achieving controlled or sustained delivery of an active composition. The compositions are especially useful for local, parenteral, buccal, gingival, and oral controlled release of active compositions, such as pharmaceuticals, and take the form of granules, encapsulated capsules, tablets, chewable gums, ingestible and implantable boluses, candies, lolipops, pourable liquids, gels, suppositories and the like.

U.S. Pat. No. 6,143,948, to Leitao, et al., “Device for incorporation and release of biologically active agents,” describes an implantable device coated with a layer of calcium phosphate and optionally one or more biologically active substances such as growth factors, lipids, (lipo)polysaccharides, hormones, proteins, antibiotics or cytostatics. The device can be obtained by a nanotechnology process comprising subjecting a substrate to a surface treatment until a surface roughness with an average peak distance (Ra value) between 10 and 1,000 nm and subjecting the roughened surface to precipitation of calcium phosphate from a solution containing calcium and phosphate ions with optional coprecipitation of the biologically active substance. The implant may be used for biomedical use, i.e. as a bone substitute, a joint prosthesis, a dental implant (prosthodontics), a maxillofacial implant, and the like.

### SUMMARY OF THE INVENTION

According to one aspect of the present invention, there is provided a device for controlled drug release, comprising:

a reservoir containing a drug; and

an electronic drug release mechanism, for providing the controlled drug release,

the device being adapted for insertion to an oral cavity of a subject.

According to an additional aspect of the present invention, the device is adapted to be removably inserted to the oral cavity of the subject.

According to an alternative aspect of the present invention, the device is adapted to be permanetly inserted to the oral cavity of the subject.

According to an additional aspect of the present invention, the device is adapted to be permanetly inserted to the oral cavity of the subject, and the device further includes a removable component, which houses at least one of the drug reservoir and the power source.

According to an additional aspect of the present invention, the electronic drug release mechanism further includes:

a control unit, for controlling the controlled release;

an electromechanical release mechanism, which opens to allow the release of the drug, responsive to commands from the control unit; and

a power source, for powering the control unit and electromechanical release mechanism.

According to an additional aspect of the present invention, the control unit is selected from the group consisting of a dedicated electronic circuitry, a processor, an ASIC, and a microcomputer.

According to an additional aspect of the present invention, the device for controlled drug release further includes a timing device, selected from the group consisting of a timer, a clock, a calendar, and a combination thereof.

According to an additional aspect of the present invention, the device further includes at least one local sensor, integrated with the device.

According to an additional aspect of the present invention, the device further includes at least two local sensors, integrated with the device.

According to an additional aspect of the present invention, the at least one local sensor is a physiological sensor, for drug release responsive to measurements of the sensor.

According to an additional aspect of the present invention, the local physiological sensor is selected from the group consisting of a sensor for drug concentration in the saliva, a sensor for glucose concentration in the saliva, a sensor for a metabolite concentration in the saliva, a sensor for an electrolyte concentration in the saliva, a sensor for the pH level in the saliva, a sensor for the temperature in the oral cavity, a heartbeat sensor, a heart rate sensor, and a snoring sensor.

According to an additional aspect of the present invention, the at least one local sensor is a status sensor, for ensuring that the device is in proper operating condition.

According to an additional aspect of the present invention, the local status sensor is selected from the group consisting of a sensor for remaining drug in the drug reservoir, a sensor for drug flow rate, a sensor for power source condition, and a sensor for short-circuit detection.

According to an additional aspect of the present invention, the device further includes at least one communication component, selected from the group consisting of a receiver, a transmitter, and a transceiver.

According to an additional aspect of the present invention, the communication component provides communication with a personal extracorporeal system.

According to an additional aspect of the present invention, the personal extracorporeal system is selected from the group consisting of a remote control unit, a computer system, a telephone, a mobile phone, a palmtop, a PDA, a laptop, and a combination thereof.

According to an additional aspect of the present invention, the personal extracorporeal system is adapted to provide communication between the device and a monitoring center.

According to an additional aspect of the present invention, the communication component provides communication with at least one remote sensor.

According to an additional aspect of the present invention, the remote sensor is selected from the group consisting of a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, a temperature sensor, a heartbeat sensor, a heart rate sensor, and a snoring sensor.

According to an additional aspect of the present invention, the device further includes at least one drug-transfer component for increased drug transfer through a biological barrier, by a process selected from the group consisting of iontophoresis, electroosmosis, electrophoresis, electroporation, sonophoresis, and ablation.

According to an additional aspect of the present invention, the drug release mechanism provides the controlled drug release in a manner selected from the group consisting of release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system.

According to an additional aspect of the present invention, the device further includes at least two drug reservoirs.

According to an additional aspect of the present invention, the drug is in nano-size particles.

According to an additional aspect of the present invention, the device is mounted on a dental implement, designed for the oral cavity of the subject.

According to an additional aspect of the present invention, the dental implement is selected from the group consisitng of a prosthetic tooth crown, a dental bridge, a dental three-unit bridge, dental implant, partial dentures, full dentures, braces, a molar band, a night guard, and a mouth guard.

According to an alternative aspect of the present invention, the device is mounted on an anchor that may be secured to the oral mucosa or the jawbone.

According to an alternative aspect of the present invention, the device is anchor-free, and is directly implanted into a tissue.

According to one aspect of the present invention, there is provided a method of controlled drug release, comprising:

providing a device for controlled drug release, which comprises a reservoir containing a drug and an electronic drug release mechanism for controllably releasing the drug; and

inserting the device in an oral cavity of a subject.

According to another aspect of the present invention, there is provided a device for controlled drug release, comprising:

a reservoir containing a drug; and

a dental implement, designed for insertion to the oral cavity of a subject, and adapted for supporting the drug reservoir.

According to an additional aspect of the present invention, the dental implement is selected from the group consisitng of a prosthetic tooth crown, a dental bridge, a dental three-unit bridge, dental implant, partial dentures, full dentures, braces, a molar band, a night guard, and a mouth guard.

According to an additional aspect of the present invention, the dental implement is designed to be removably inserted to the oral cavity of the subject.

According to an alternative aspect of the present invention, the dental implement is designed to be permanently inserted to the oral cavity of the subject.

According to an additional aspect of the present invention, the dental implement is designed to be permanetly inserted to the oral cavity of the subject, and the dental implement further includes a removable component, which houses at least one of the drug reservoir and the power source.

According to another aspect of the present invention, there is provided a method of controlled drug release, comprising:

providing a device for controlled drug release, which comprises a reservoir containing a drug; and

supporting the device in an oral cavity of a subject, on a dental implement, designed for insertion to the oral cavity of a subject and for supporting said device.

The present invention successfully addresses the shortcomings of the presently known configurations by providing drug dosage forms, which are housed in oral devices, and methods for controlled drug release. The oral devices are permanently or removably inserted in the oral cavity and refilled or replaced as needed. The controlled drug release may be passive, based on the dosage form, or electronically controlled, for a high-precision, intelligent, drug delivery. Additionally, the controlled release may be any one of the following: release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system. Drug absorption in the oral cavity may be assisted by an electrotransport mechanism. The oral devices require refilling or replacement at relatively long intervals of weeks or months, maintain a desired dosage level in the oral cavity, hence in the gastrointestinal tract, for extended periods, address situations of narrow drug therapeutic indices, and by being automatic, ensure adherence to a prescribed medication regimen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a cross-sectional view of a tooth, as known;

FIGS. 2A-2G schematically illustrate the steps in root canal therapy, as known;

FIGS. 3A-3F schematically illustrate the application of a dental bridge, as known;

FIGS. 4A-4C schematically illustrate the application of a dental implant, as known;

FIGS. 5A-5C schematically illustrate the dentures, as known;

FIGS. 6A-6C schematically illustrate the application of a dental crown, as known;

FIGS. 7A-7C schematically illustrate the braces, as known;

FIGS. 8A-8D schematically illustrate dental bridges, which include devices for controlled drug release, in accordance with preferred embodiments of the present invention;

FIGS. 9A-9I schematically illustrate a dental bridge, which includes an electronic device for controlled drug release, in accordance with another preferred embodiment of the present invention;

FIG. 10 schematically illustrates a dental implant, which includes an electronic device for controlled drug release, in accordance with still another preferred embodiment of the present invention;

FIGS. 11A-11D schematically illustrate dentures, which include at least one device for controlled drug release, in accordance with another preferred embodiment of the present invention;

FIGS. 12A-12H schematically illustrate dental braces, which include at least one device for controlled drug release, in accordance with another preferred embodiment of the present invention; and

FIGS. 13A-13D are schematic diagrams of electronic devices for controlled drug release, in accordance with some preferred embodiments of the present invention.

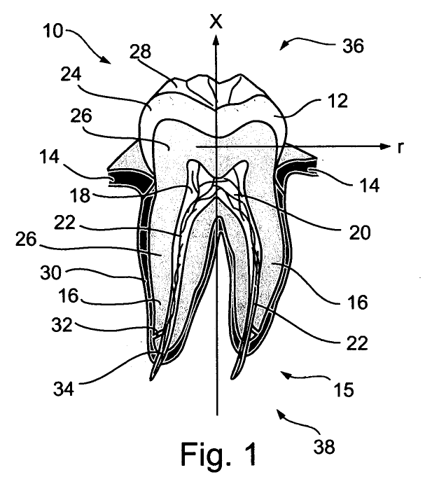

### DESCRIPTION OF THE PREFERRED EMBODIMENTS

The present invention is of drug dosage forms, which are housed in oral devices, and of methods for controlled drug release. The oral devices are permanently or removably inserted in the oral cavity and refilled or replaced as needed. Specifically, the controlled drug release may be passive, based on the dosage form, or electronically controlled, for a high-precision, intelligent, drug delivery. Additionally, the controlled release may be any one of the following: release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system. Drug absorption in the oral cavity may be assisted by an electrotransport mechanism. The oral devices require refilling or replacement at relatively long intervals of weeks or months, maintain a desired dosage level in the oral cavity, hence in the gastrointestinal tract, for extended periods, address situations of narrow drug therapeutic indices, and by being automatic, ensure adherence to a prescribed medication regimen.

The principles and operation of the substance and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

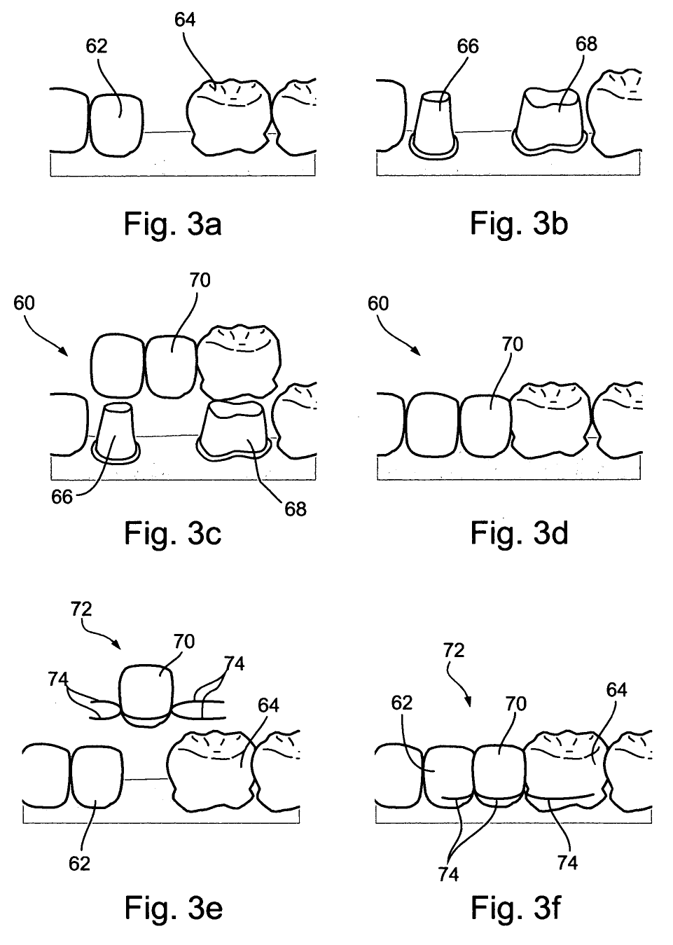

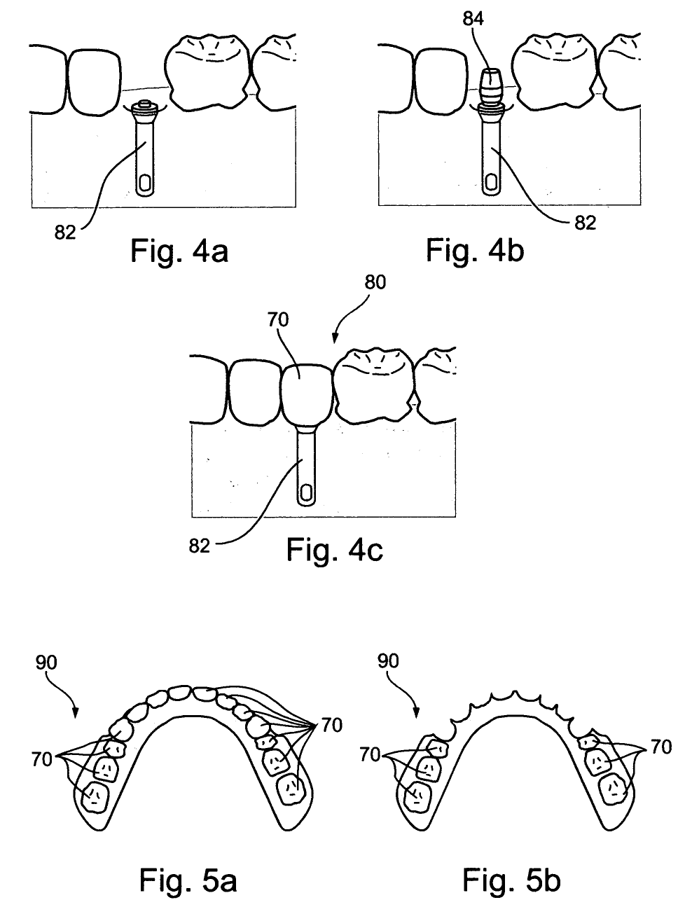

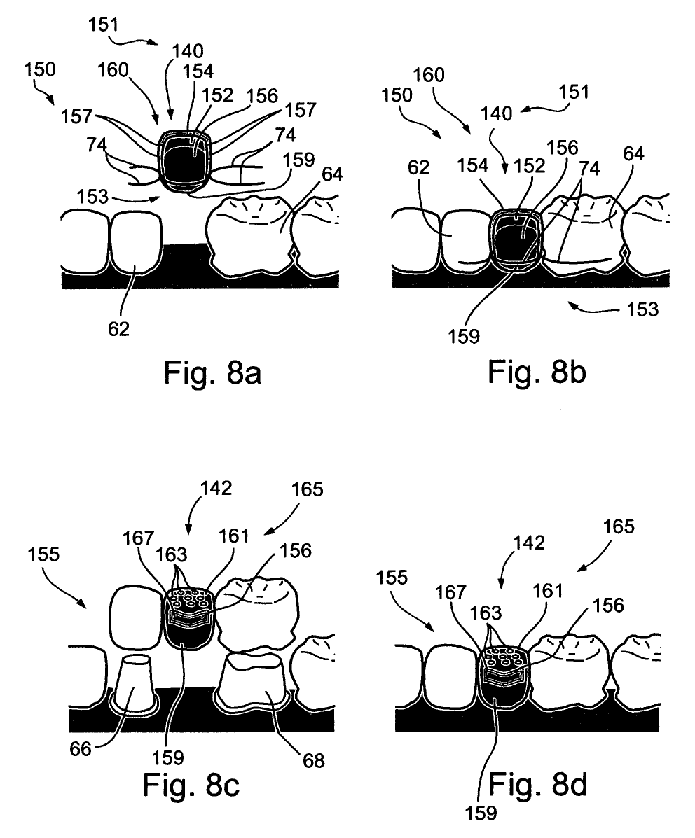

Referring now to the drawings, FIGS. 8A-8B schematically illustrate a device 140, for controlled drug release, mounted on a dental bridge 150, in accordance with a preferred embodiment of the present invention. Preferably, dental bridge 150 is removable, constructed in the manner taught in FIGS. 3E-3F, hereinbelow.

Device 140, for controlled drug release, is designed as a prosthetic tooth crown 160, and mounted on dental bridge 150, for insertion in the gap between teeth 62 and 64, with clamps 74. Preferably, impressions of teeth 62 and 64 and the gap between them have been made, and dental bridge 150 with prosthetic tooth crown 160 are adapted for a specific patient. Prosthetic tooth crown 160 preferably includes a hard outer shell 154, for example, of metal or porcelain, having a coronal side 151 and an apical side 153, wherein the coronal surface is adapted for chewing.

An inner space of prosthetic tooth crown 160 includes a drug reservoir 156, in a dosage form adapted for passive, controlled release. As used herein, passive drug release relates to controlled release, which is not governed by an electronic device. Passive drug release includes for example, the methods of dosage form preparation described hereinbelow, in items 1-14.

Preferably, hard outer shell 154 includes at least one, and preferably several perforations 157 for the drug release. Additionally or alternatively, a semi-pervious membrane 159 may be used, for example on apical side 153. In accordance with the present invention, one or several perforations 157 and (or) semi-pervious membrane 159, may be operative in the controlled release of the drug. Where necessary, filler 152 may be used around the drug reservoir. Once placed in the oral cavity, the drug is released to the oral cavity and (or) oral tissue, in a controlled manner, by a natural phenomenon.

Two or more dental bridges 150 may be prepared for a patient, in order to maintain a steady supply of drug as the device is being refilled. Alternatively, a single dental bridge 150 may be used, arranged for on-the-spot, quick refilling.

The key advantage of device 140 is that unlike ingested dosage forms, which may maintain a predetermined therapeutic drug concentration in the plasma for about 12 hours, before they are absorbed or eliminated by the gastrointestinal tract, orally implanted dosage forms may maintain a predetermined therapeutic drug concentration for periods of months. As such, the oral implanted dosage forms offers a variable alternative to gastro-retention devices.

Several controlled release mechanisms may be used, for example, as taught by Encyclopedia of Controlled Drug Delivery, volume 2, edited by Edith Mathiowitz, pp. 838-841. These are based on the use of specific substances, generally polymers, as a matrix or as a coating, which degrade fast or slowly, depending on the desired effect. Yet, while the Encyclopedia of Controlled Drug Delivery generally considers the gastrointestinal fluids as the ambient solvent, in accordance with the present invention, saliva, whose pH value is about 5.2-6.8 is the ambient solvent. In accordance with the present invention, the drug of reservoir 156 may be a in a dosage form for passive, controlled release, prepared by any one of the following methods:

1. The drug, which may be solid, liquid or a suspension in liquid, may be encapsulated in a polymeric material, so that the drug release is controlled by diffusion through the capsule walls.

2. The drug particles may be coated with wax or poorly soluble material, or an insoluble material (e.g., plyvinyl chloride) mixed with a soluble, pore forming compound, so that the drug release from reservoir 156 is controlled by the breakdown of the coating.

3. The drug may be embedded in a slow-release matrix, which may be biodegradable or non-biodegradable, so that the drug release from reservoir 156 is controlled by diffusion through the matrix, erosion of the matrix, or both.

4. The drug may be complexed with ion-exchange resins that slow down its release.

5. The drug may be laminated, as a jellyroll, with a film, such as a polymeric material, which may be biodegradable or nonbiodegradable, so that the drug is released by diffusion, erosion or both.

6. The drug may be dispersed in a hydrogel, or a substance that forms a hydrogel in the oral cavity, so that the drug release from reservoir 156 is controlled by diffusion of the drug from the water-swollen hydrogel.

7. Osmotic pressure may be used to release the drug in a controlled manner—uptake of water into reservoir 156 may increase the osmotic pressure within reservoir 156. The build up of the osmotic pressure will drive the drug through one or more orifices to release the drug in a controlled manner.

8. The drug may be chemically bonded to a polymer and released by hydrolysis.

9. Macromolecular structures of the drug may be formed via ionic or covalent linkages, which control the drug release from reservoir 156 by hydrolysis, thermodynamic dissociation or microbial degradation.

10. The drug may be coated with a combination of a soluble and insoluble polymers; when the soluble particles dissolve, they will form a microporous layer around the drug core, so that the drug may permeate slowly through the micropores; the rate of release depending on the porosity and thickness of the coating layer.

11. The drug may be designed for pH independent controlled release, and produced by wet granulating an acidic or basic drug blend with a buffering agent and the appropriate excipients, wherein the granules are then compressed into tablets, which are further coated with a film permeable to the saliva. Upon oral administration, saliva permeates the film coating, at which time the buffering agents adjust the pH value of the tablet so that the drug can dissolve and permeate out of the dosage form at a constant rate, independent of the pH level in the mouth.

12. The dosage formulation may be sealed in the non-soluble capsule body by means of a water soluble plug and a hydrogel plug. When the capsule is placed in the oral cavity, the water-soluble cap dissolves and exposes the hydrogel plug, which begins to swell. At a predetermined time after placement, the hydrogel plug is ejected and the encapsulated dosage formation is released.

13. Multiparticulate dosage forms may be used. Sugar or nonpareil pellets may be spray coated with a drug, dried, then spray coated with a second coating composition, which provides controlled release. The second coating composition is typically formed of polymers, which are partially soluble or insoluble in the saliva, wherein the degree of solubility depends on the desired drug release pattern. The doubly coated pellets are placed in a capsule. A capsule can contain pellets of different types and release profiles.

14. A dosage form of nano-size particles may be used, for improved solubility.

Referring now to the drawings, FIGS. 8C-8D schematically illustrate a device 142, for passive, controlled drug release, mounted on a three-unit bridge 155, analogous to that taught in FIGS. 3A-3D, hereinbelow, in accordance with another preferred embodiment of the present invention.

As seen in FIGS. 3A-3B, hereinbelow, the dentist prepares teeth 62 and 64 on either side of a gap by removing portions of the enamel and dentin, leaving stumps 66 and 68. Impressions or molds of stumps 66 and 68 and of the gap between them are taken for the construction of bridge 155.

As seen in FIGS. 8C-8D, three-unit bridge 155 includes device 142, for passive, controlled drug release, designed as a prosthetic tooth crown 165. Prosthetic tooth crown 165 has a hard outer shell 161, for example, of metal or porcelain, adapted as a chewing surface. Hard outer shell 161 includes a removable component, such as a drawer 167, for refilling, or for replacement. Drawer 167 includes drug reservoir 156, in a dosage form adapted for passive, controlled release, similar, for example, to that of FIGS. 8A-8B. Preferably, hard outer shell 161 includes at least one, and preferably several or a plurality of perforations 163 for the drug release, or another manner of opening, for the drug release. Additionally or alternatively, semi-pervious membrane 159 may be used. Once placed in the oral cavity, the drug is released to the oral cavity and (or) oral tissue, in a controlled manner, by a natural phenomenon.

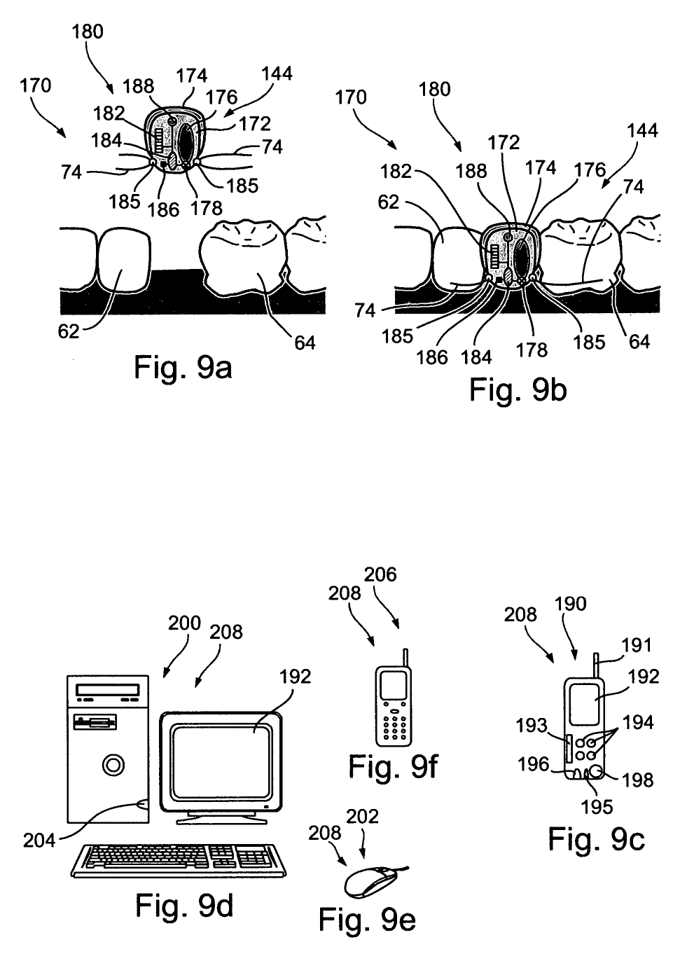

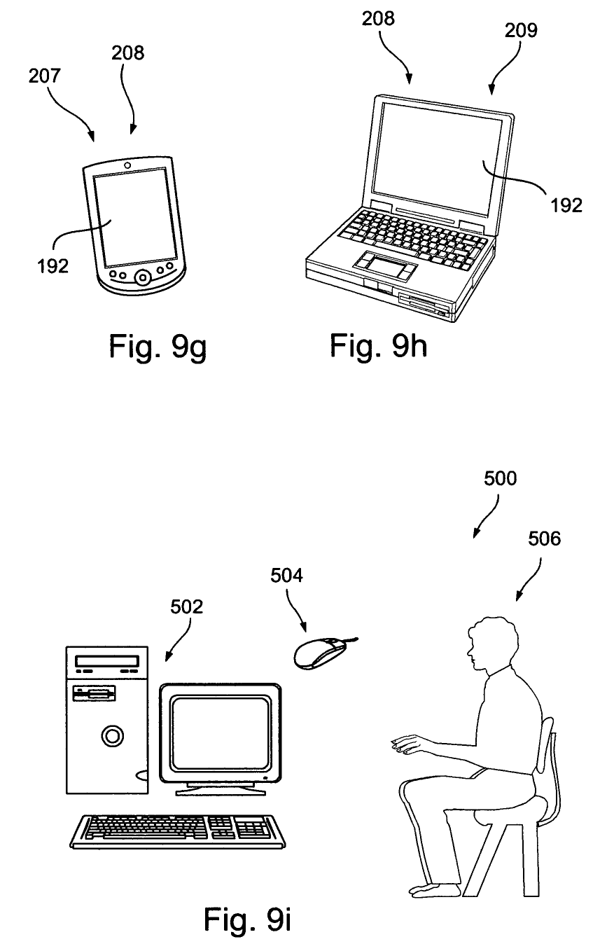

Referring further to the drawings, FIGS. 9A-9I schematically illustrate a device 144 for electronic, controlled drug release, mounted on a dental bridge 170, in accordance with another preferred embodiment of the present invention.

As seen in FIGS. 9A and 9B, dental bridge 170 is preferably, removable, constructed in the manner taught in FIGS. 3E-3F, hereinbelow.

Device 144 for electronic, controlled drug release is designed as a prosthetic tooth crown 180, mounted on dental bridge 170, for insertion in a gap between teeth 62 and 64, with clamps 74. Preferably, dental bridge 170 is adapted for a specific patient. Prosthetic tooth crown 180 preferably includes a hard outer shell 174, adapted as a chewing surface. Two or more dental bridges 170 may be prepared for a patient, in order to maintain a steady supply of drug as the device is being refilled. Alternatively, a single dental bridge 170 may be used, arranged for on-the-spot, quick refilling.

An inner space of prosthetic tooth crown 180 is designed as a device for electronic, controlled drug release, for high-precision, intelligent drug delivery. The electronics may be encased within filler 172, for example, silicon. Prosthetic tooth crown 180 includes a drug reservoir 176, having an orifice controlled by an electro-mechanical release mechanism, such as a solenoid 178. A power source 182 provides prosthetic tooth crown 180 with power. A control unit 184 controls the operation of electromechanical release mechanism 178, for the issuance of drug to the oral cavity and (or) oral tissue, in a controlled manner. Control unit 184 may be any one of a dedicated control circuitry 184, a processor 184, an Application Specific Integrated Circuit (ASIC) 184, or a microcomputer 184, as known, and may further include built-in intelligence. A memory unit 186 may be integrated with it. It will be appreciated that control unit 184 may control both the timing for drug release and the release rate. It will be appreciated that power source 182 may be any power source, for example, a battery or a solid-electrolyte fuel cell.

Preferably, control unit 184 has a built-in timing device, which preferably includes a timer, a clock and a calendar, and is operative to perform chronotherapy.

Additionally, a receiver 188, which may further operate as a transceiver, provides communication with a personal extracorporeal system 208, for example, as described in conjunction with FIGS. 9C-9H. It will be appreciated that a separate transmitter may be used. Transceiver 188 may operate by RF, IR or ultrasound. It may further utilize Bluetooth protocol. (A short-range communication protocol, within a range of about 3 meters.)

Device 144 may further include at least one and preferably several sensors 185, incorporated to device, and thus termed “local sensors,” to distinguish them from remote sensors, located elsewhere in the body. Local sensors 185 may be divided into two groups:

i. physiological sensors 185, for measuring, for example, a drug concentration in the saliva, glucose concentration in the saliva, a metabolite concentration in the saliva, an electrolyte concentration in the saliva, the pH level in the saliva, the temperature in the oral cavity, and any other physiological parameter or parameters, preferably having a bearing on the drug release schedule; and

ii. status sensors 185, for ensuring that the device is in proper operating condition, for example, by measuring the amount of drug remaining in the drug reservoir, the drug flow rate, the power source condition, a short circuit, or any other information relevant to the proper operation of device 144 for electronic, controlled drug release.

Physiological sensor 185 may be, for example, an electrochemical glucose sensor, such as a enzymatic biosensor taught in http://www.cfdrc.com/applications/biotechnology/biosensor.html, which utilizes the biospecificity of an enzymatic reaction, along with an electrode reaction that generates an electric current or a potential difference for quantitative analysis. The enzymatic oxidation of glucose produces hydrogen peroxide, which in turn generates electrons by electrode reaction. The current density is used as a measure of glucose in a sample, for example, in interstitial fluid.

Additionally or alternatively, glucose levels may be monitored for example, as taught by U.S. Pat. No. 6,201,980, to Darrow, et al., dated Mar. 13, 2001, entitled, “Implantable medical sensor system,” whose disclosure is incorporeated herein by reference. Darrow, et al. disclose an implantable chemical sensor system for medical applications, which permits selective recognition of an analyte using an expandable biocompatible sensor, such as a polymer, that undergoes a dimensional change in the presence of the analyte. The expandable polymer is incorporated into an electronic circuit component that changes its properties (e.g., frequency) when the polymer changes dimension. As the circuit changes its characteristics, an external interrogator transmits a signal transdermally to the transducer, and the concentration of the analyte is determined from the measured changes in the circuit. The implantable chemical sensor system may be used for minimally invasive monitoring of blood glucose levels or interstitial fluid glucose levels in diabetic patients.

Additionally or alternatively, physiological sensors 185 may be, for example, as taught by U.S. Pat. No. 6,058,331, to King, dated May 2, 2000, and entitled, “Apparatus and method for treating peripheral vascular disease and organ ischemia by electrical stimulation with closed loop feedback control,” whose disclosure is incorporated herein by reference. King discloses techniques for therapeutically treating peripheral vascular disease, wherein a sensor is employed for sensing the extent of blood flow in a patient's limb or ischemic pain and generating a response, based on the sensor's reading.

Alternatively, physiological sensors 185 may be based on Ambri's Ion Channel Switch (ICS™) technology of biosensors of a self assembling synthetic bio-membrane, as described in http://www.ambri.com/Content/display.asp?screen=174. It is one of the world's first true ‘bio-nano’ devices. Ambri has built a biological switch: a membrane, which can detect the presence of specific molecules and signal their presence by triggering an electrical current. This device—the Ambri Ion Channel Switch(ICS™) Biosensor—is a two molecular layer self assembled membrane based on the ion channel gramicidin.

As taught by PCT publication W0 0174446, to Karachurov, a plurality of miniature sensors of a same type may be employed, to increase the accuracy of the measurements. Additionally or alternatively, sensors of different types may be used. Furthermore, several sensor modules 185 may be employed, at different locations in the body.

Device 144 may further include at least one, and preferably several remote physiological sensors 185, implanted or otherwise placed elsewhere in the body, each having its own power supply and transmitter or transceiver. Additionally or alternatively, a remote sensor module 185 of several physiological sensors, possibly of different types, may be employed, wherein the several sensors share a power supply, a transmitter or transceiver, and possibly a control unit. The remote sensor module may further include a remote status sensor 185, for reporting the remote-sensor power source condition.

Examples of remote physiological sensors 185 may include a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, a temperature sensor, a heartbeat sensor, a heart rate sensor, and a snoring sensor.

Remote sensors 185 may be intracorporeal, implanted under the skin, for example, in the chest or under the arm, for measuring, for example, interstitial fluid drug concentration level, interstitial fluid glucose level, tissue temperature, and heart rate. Additionally or alternatively, remote sensors 185 may be intracorporeal, implanted on stents, in blood vessels, for measuring, for example, blood drug concentration level, blood glucose level, or blood oxygen level.

Additionally or alternatively, remote sensors 185 may be extracorporeal, for example, attached to the skin. The extracorporeal sensors may include piezoelectric patches that may be attached to the skin, by adhesives, for measuring heart rate, patches for measuring body temperature, and (or) sensors that measure concentration levels of the drug, or of other chemicals, such as glucose, in the sweat.

For example, extracorporeal, remote sensors 185 may be similar to those taught by Lin, G., and Tang, W., “Wearable Sensor Patches for Physiological Monitoring,” NASA's Jet Propulsion Laboratory, Pasadena, Calif., which may be found at http://www.nasatech.com/Briefs/Feb00/NPO20651.html, or in NASA Tech Briefs: NPO-20651, which may be obtained from Technology Reporting Office, JPL, Mail Stop 122-116, 4800 Oak Grove Drive, Pasadena, Calif. 91109, (818) 354-2240. The wearable sensor patches, formed as miniature biotelemetric units, may be employed for measuring temperature, heart rate, blood pressure, and possibly other physiological parameters. The sensor patches are designed small and may be mass-produced inexpensively by use of state-of-the-art techniques for batch fabrication of integrated circuits and microelectromechanical systems. Each patch may be a few centimeters on a side, comparable in size to an ordinary adhesive bandage. The patch may even be held on the wearer's skin by the same adhesive as that used on bandages. The patch may contain a noninvasive microelectromechanical sensor integrated with electronic circuitry operative to process the sensor output and transmit a radio signal modulated by the processed sensor output.

As for the local sensors, a plurality of miniature sensors of a same type may be employed, to increase the accuracy of the measurements. Additionally or alternatively, sensors of different types may be used. Furthermore, several sensor modules 185 may be used, at different locations in the body.

Communication between remote sensors 185 and prosthetic tooth crown 180 of device 144 is preferably by ultrasound, but may be by IR or RF, and may employ communication protocols, such as Bluetooth. Additionally or alternatively, remote sensors 185 may communicate with one or more personal extracorporeal systems 208, described in conjunction with FIGS. 9C-9H, hereinbelow, preferably by IR or RF, and may employ communication protocols, such as Bluetooth. Communication may be on a continuous basis, at intervals, in reply to interrogation, or when a sudden change in a measured physiological parameter is observed.

In accordance with some embodiments, the remote sensors do not have power sources, but respond to interrogation, which further provides them with power for measuring and responding, as known.

FIGS. 9C-9H describe various personal extracorporeal systems 208 that may communicate with prosthetic tooth crown 180 and possibly also with sensors or sensors 185, with each other, and with a monitoring center, described in conjunction with FIG. 9I. Communication between personal extracorporeal systems 208 may be performed via connectors 196 and cables, for example, via UBS connectors, or by RF or IR waves, for example, using Bluetooth protocol. Personal extracorporeal systems 208 are termed “personal” as they may be on the premises of the patient, to distinguish them from the monitoring center.

As seen in FIG. 9C, personal extracorporeal system 208 may be a remote-control unit 190, which may include a display panel 192, control buttons 194, a connector 196 for connection to a computer system, preferably being a UBS connector, a transmitter 198, which may further operate as a transceiver 198, preferably, an antenna 191, a power source 193, and preferably also a plug for recharging power source 195. It will be appreciated that a separate receiver may be used. Transceiver 198 may operate by RF, IR and may employ Bluetooth protocol.

Additionally, as seen in FIGS. 9C-9H, personal extracorporeal system 208 may be a computer system 200, a telephone 202, a mobile phone 206, a palmtop or PDA 207, a laptop 209, or another remote system, as known. In general, these personal extracorporeal systems 208 include display panel 192.

Communication to device 144 may include a demand to release drug immediately, stop the release, increase or decrease the release rate, or specify a long term or a short tem release schedule and release rate for the drug.

Communication from device 144 may include the operating release schedule and rate for the drug and indications of sensors 185, for example, drug concentration in the saliva, glucose level in the saliva, the amount of drug remaining in drug reservoir 176, drug flow rate, and a low power source indication. These measurements may be displayed on display panel 192 of any of personal extracorporeal system 208.

Either one of personal extracorporeal system 208, or prosthetic tooth crown 180 of device 144 may process the communicated measurements of sensors 185 by means of built-in intelligence and algorithms, for drug release, responsive to the communicated measurements, to compensate for the measurement, to correct a situation that is indicated by it, and (or) to improve the efficacy and to optimize drug release, for an optimal closed-loop operation. Additionally or alternatively, either personal extracorporeal system 208, or prosthetic tooth crown 180 of device 144 may process the communicated measurements of sensors 185, to calibrate the drug release with the measured data, in order to arrive at an optimal release schedule for the closed-loop operation.

As seen in FIG. 9I, a monitoring center 500 may oversee the drug administration program of device 144. Monitoring center 500 may be a clinic, a heath center, a drug rehabilitation center, or another monitoring center, as applicable. Preferably, monitoring center 500 includes an attendant 506, such as a medical practioner, a nurse, a social worker, and (or) another attentdant, as applicable, a computer system 502, and a telephone or cell phone 504. Monitoring center 500 may also be a center-on-the-go, for example, of a medical practitoner, his laptop, and his cell phone. Communication between device 144 and monitoring center 500 is preferably by any one of personal extracorporeal systems 208.

It will be appreciated that personal extracorporeal systems 208, for example, any one of, or several of telephone 202, mobile phone 206, palmtop 207 and PDA 207 may be designed with specific codes for quick and easy communication both with monitoring center 500 and with device 144. For example, dialing *10 may reach medical attendant 506 at monitoring center 500, dialing *11 may reach computer system 502 at monitoring center 500, dialing *12 may communicate with device 144 and start the release of drug, dialing *13 may also communicate with device 144 and increase the release rate of the drug. In general, personal extracorporeal systems 208 are operative as intermediaries between device 144 and monitoring center 500, forwarding to monitoring center 500 data from device 144, and to device 144, commands from monitoring center 500.

It will be appreciated that device 144 may also be a self-contained system, and operate without an extracorporeal system or any remote control.

It will be appreciated that prosthetic tooth crown 180 may also be designed on a three-unit bridge, in a manner analogous to prosthetic tooth crown 165 of FIGS. 8C-8D, wherein parts that need replacement, such as drug reservoir 176 and possibly also power source 182 are located in a drawer, analogous to drawer 167 there.

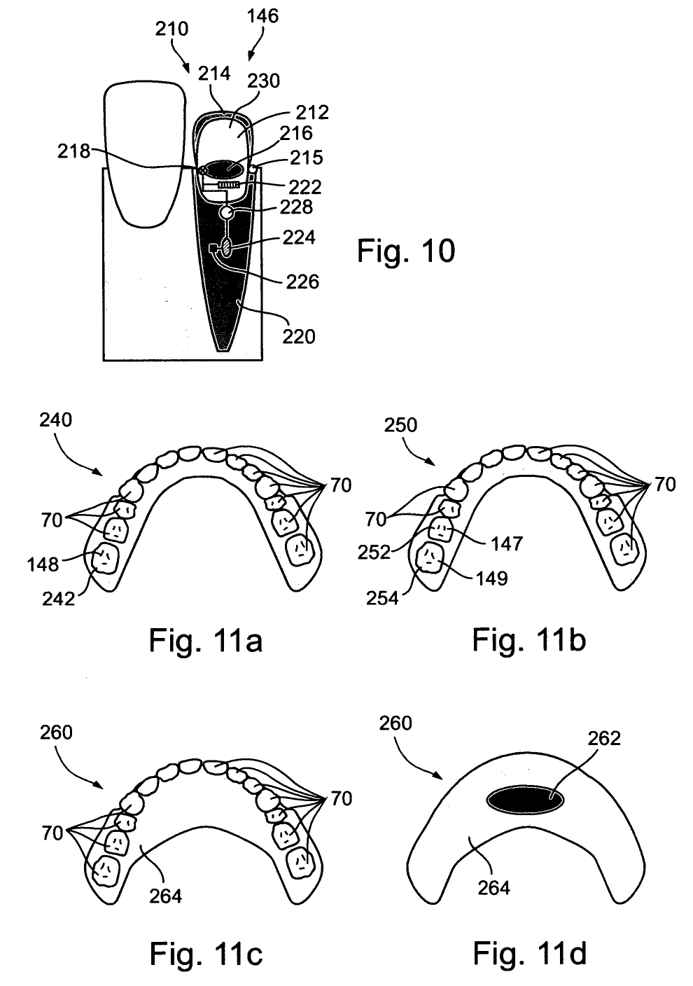

Referring further to the drawings, FIG. 10 schematically illustrates a device 146 for electronic, controlled drug release, designed as a dental-implant-and-prosthetic-tooth-crown 210, in accordance with still another preferred embodiment of the present invention. Device 146 for electronic, controlled drug release has a permanent portion 220, located in the post and a removable portion 230, in the crown. Removable portion 230, in the crown of device 146, includes a drug reservoir 216, whose drug release is controlled by an electromechanical release mechanism 218. A power source 222 provides power. These are encased within filler 212, for example silicon. A hard shell 214 provides the chewing surface. Preferably, impressions have been taken so that removable portion 230 is adapted for a specific patient. Additionally, two or more removable portions 230 may be made, so that one is in operation while the other is being refilled. Alternatively, a single removable portions 230 may be used, arranged for on-the-spot, quick refilling of drug reservoir 216 and (or) power source 222.

Permanent portion 220, in the post, may include a control unit 224, such as a processor 224, for controlling the operation of electromechanical release mechanism 218, preferably also a memory unit 226, and a transmitter-receiver 228. At least one sensor 215 may be located on the interface between the post and the crown, and may be attached to either. Alternatively, at least one sensor 215 may be located within the post or within the crown. Alternatively, the sensor or sensors may be located elsewhere in the body. Electro-mechanical release mechanism 218 may be located in the post or in the crown of device 146.

The operation of the present embodiment is similar to that of the embodiment of FIGS. 9A-9I, in conjunction with remote-control unit 190 and (or) computer system 200, save for the advantage that only the portions of the electronic device that need replacement, namely the drug reservoir and the power source, are adapted for removing.

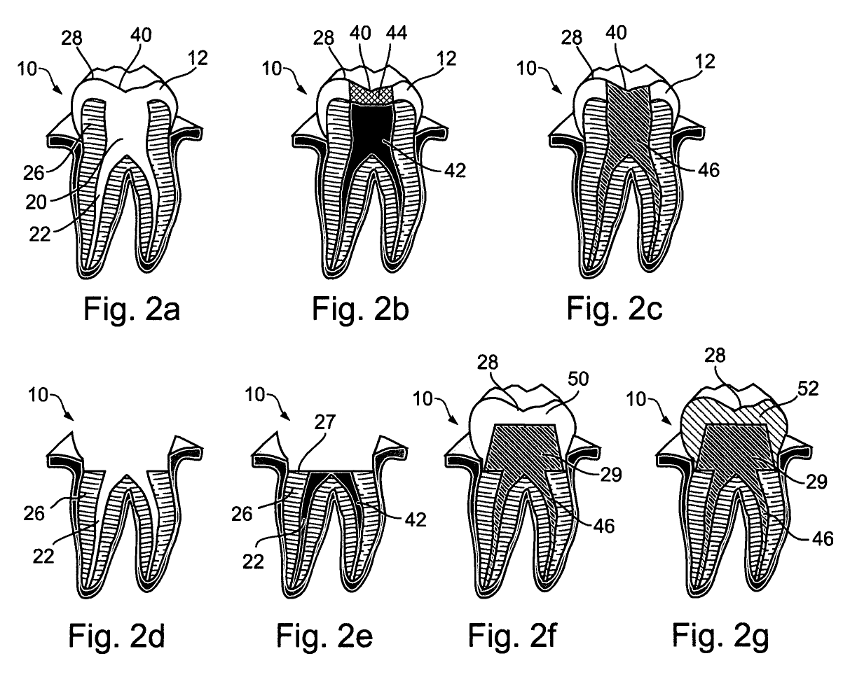

It will be appreciated that a similar construction of a permanent portion and a removable portion may be used in conjunction with a root canal (FIGS. 2A-2G). The permanent portion may be located in the canal, and the removable portion may be located in the crown.

It will be appreciated the crown of device 146 may also be designed in a manner analogous to prosthetic tooth crown 165 of FIGS. 8C-8D, wherein parts that need replacement, such as drug reservoir 176 and possibly also power source 182 are located in a drawer, analogous to drawer 167 there.

Referring further to the drawings, FIGS. 11A-11D schematically illustrate full dentures, which include at least one device for controlled drug release, in accordance with another preferred embodiment of the present invention. It will be appreciated that partial dentures may similarly be used.

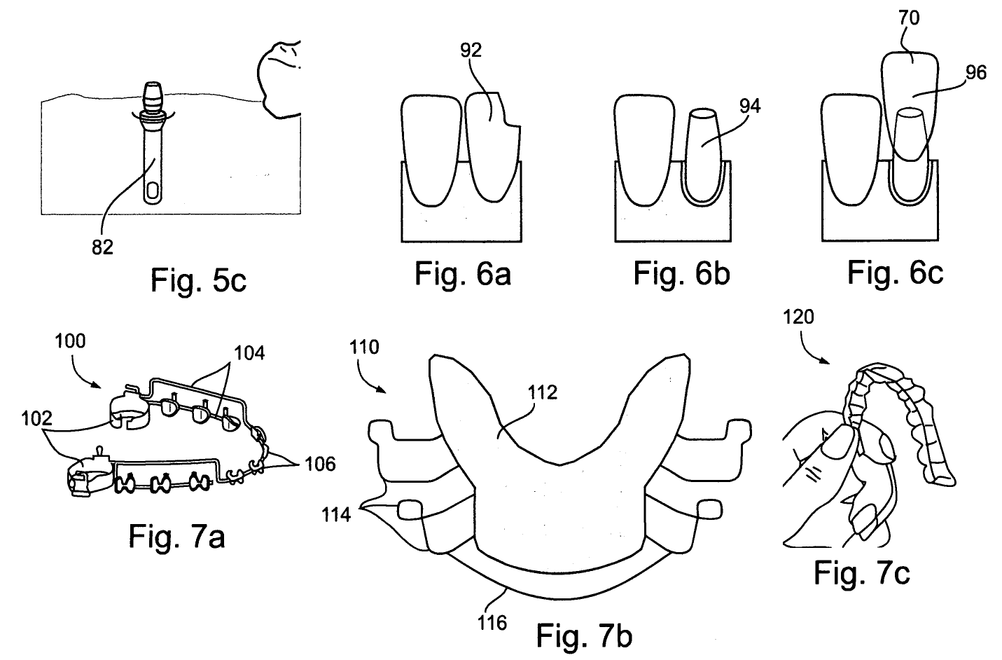

As seen in FIG. 11A, dentures 240 includes a plurality of prosthetic tooth crowns 70, as taught in conjunction with FIGS. 5A-5C, hereinbelow. Additionally, dentures 240 include a device 148 for controlled drug release, designed as a prosthetic tooth crown 242. Prosthetic tooth crown 242 may be adapted for passive controlled drug delivery, as taught in conjunction with FIGS. 8A-8D. Alternatively, prosthetic tooth crown 242 may be adapted for electronically controlled drug delivery, as taught in conjunction with FIGS. 9A-9B, and preferably operate with any one of or a combination of personal extracorporeal systems 208, described in conjunction with FIGS. 9C-9H, and with monitoring center 500 of FIG. 9I.

As seen in FIG. 11B, dentures 250 includes a plurality of prosthetic tooth crown 70, as taught in conjunction with FIGS. 5A-5C, hereinbelow. Additionally, dentures 250 include devices 147 and 149, designed as prosthetic tooth crowns 252 and 254, for controlled drug release. These may be adapted for passive controlled drug delivery, as taught in conjunction with FIGS. 8A-8D, or for electronically controlled drug delivery, as taught in conjunction with FIGS. 9A-9B, and preferably operate with any one of or a combination of personal extracorporeal systems 208, described in conjunction with FIGS. 9C-9H, and with monitoring center 500 of FIG. 9I.

Additionally, more than two prosthetic tooth crowns for controlled drug delivery may be employed.

Alternatively, prosthetic tooth crowns 252 and 254 may form a single device for electronically controlled drug delivery, wherein prosthetic tooth crown 252 may form a removable portion, which includes the drug reservoir and power source, which must be replaced periodically, while prosthetic tooth crown 254 may include the permanent components, as taught in conjunction with FIG. 10, hereinbelow.

FIGS. 11C and 11D illustrate front and back sides of full dentures 260, which include a plate 264, which may be fitted under the tongue, for bottom dentures, or against the roof of the mouth, for top dentures. The backside (FIG. 11D) further includes a device 262, for controlled drug release. In this manner, buccal and sublingual administration may be enhanced. The advantage of these types of administration is that they lead to direct absorption to the blood stream, avoiding the GI route and the liver.

Device 262 for controlled drug release may be passive or electronically controlled.

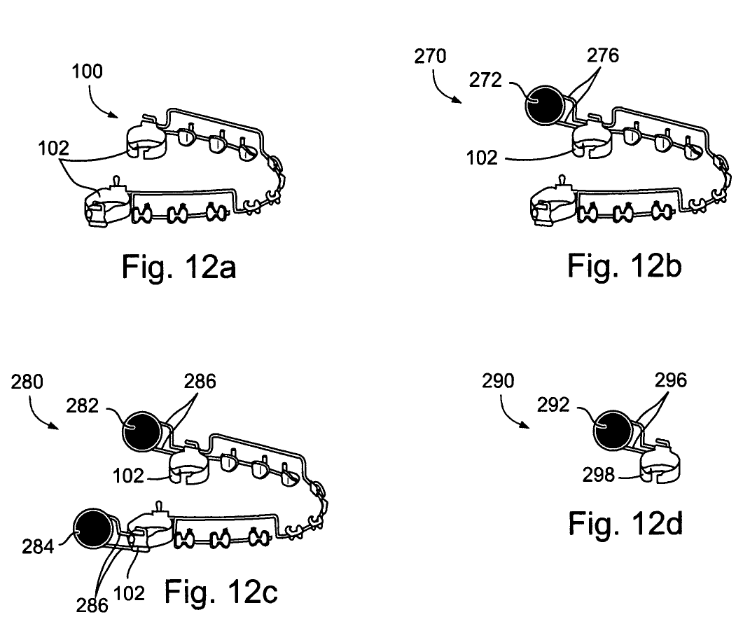

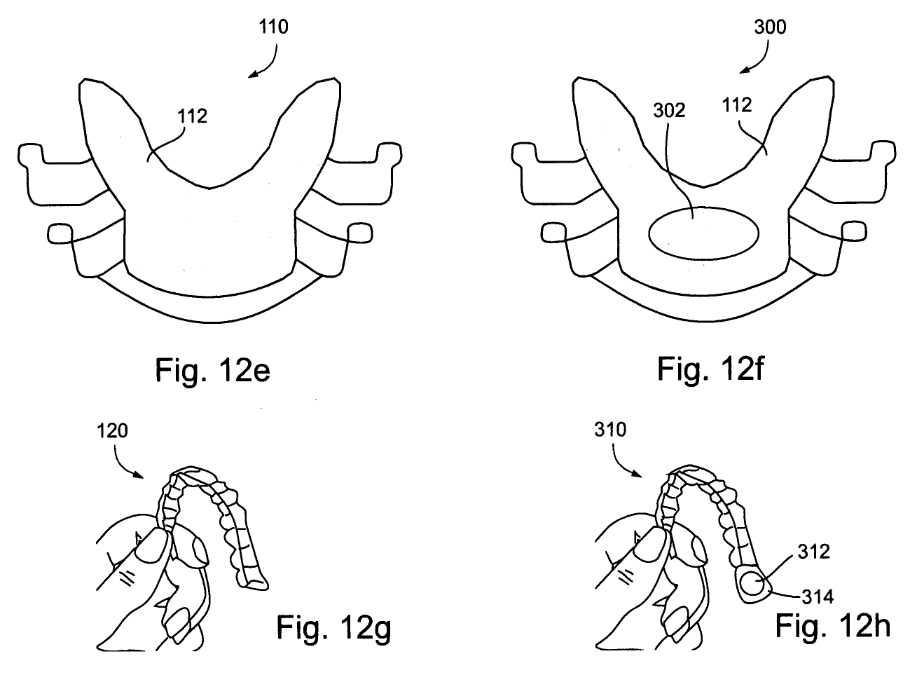

Referring further to the drawings, FIGS. 12A-12H schematically illustrate dental braces, which include at least one device for controlled drug release, in accordance with another preferred embodiment of the present invention.

While FIG. 12A schematically illustrates conventional braces 100, having molar bands 102, as taught in conjunction with FIG. 7A, hereinbelow, FIG. 12B illustrates braces 270, which include a device 272 for controlled drug release, in accordance with a preferred embodiment of the present invention. Device 272 is attached to molar bands 102 with wires 276.

Additionally, FIG. 12C illustrates braces 280, which include devices 282 and 284, for controlled drug release, in accordance with a preferred embodiment of the present invention. Devices 282 and 284 are attached to molar bands 102 with wires 286. Additional devices may similarly be employed.

Furthermore, FIGS. 12D illustrates an arrangement 290, in which a device 292 for controlled drug release, is attached to a molar band 298, with wires 296, in accordance with a preferred embodiment of the present invention.

While FIG. 12E schematically illustrates conventional braces 110, having a plate 112, as taught in conjunction with FIG. 7B, hereinbelow, FIG. 12F illustrates braces 300, which include a device 302 for controlled drug release, arranged on the back side of plate 112, in accordance with a preferred embodiment of the present invention. Thus, device 302 is adapted for enhanced buccal and sublingual administration.

While FIG. 12G schematically illustrates conventional invisible braces 120, as taught in conjunction with FIG. 7C, hereinbelow, FIG. 12H illustrates braces 310, which include a device 312 for controlled drug release, arranged on an added invisible portion 314. In a similar manner, a mouth guard or a night guard may be used, for attaching a device for controlled drug release.

It will be appreciated that since braces are generally employed by children whose wisdom teeth have not yet emerged, the space generally occupied by the wisdom teeth may be used for the extensions shown in FIGS. 12B-12D and 12H.

Devices 272, 282, 284, 292, 302 and 312 for controlled drug release may be passive or electronically controlled.

In accordance with the present invention, the devices for electrically controlled drug release may further include at least one drug-transfer component for increased drug transfer through a biological barrier, to enhance buccal and sublingual direct absorption. The drug transfer mechanism may include iontophoresis, electroosmosis, electrophoresis, electroporation, sonophoresis, and ablation. The at least one drug-transfer component may be, for example, at least one electrode or several electrodes, for an electrotransport mechanism including electric ablation, an ultrasound transducer, for sonophoresis, a microwave coil, for microwave ablation, an RF coil, for RF ablation, or a laser diode, for laser ablation, as known. Additionally, a combination of these may be employed. These mechanisms may be controlled by control unit 184 (FIGS. 9A-9B). Additionally or alternatively, they may be controlled remotely, by personal extracorporeal system 208 (FIGS. 9C-9H), such as remote-control unit 190, computer system 200, telephone 202, mobile phone 206, palmtop 207, laptop 209, or any other remote-control unit, as known. Additionally or alternatively, they may be controlled by monitoring center 500, via personal extracorporeal system 208.

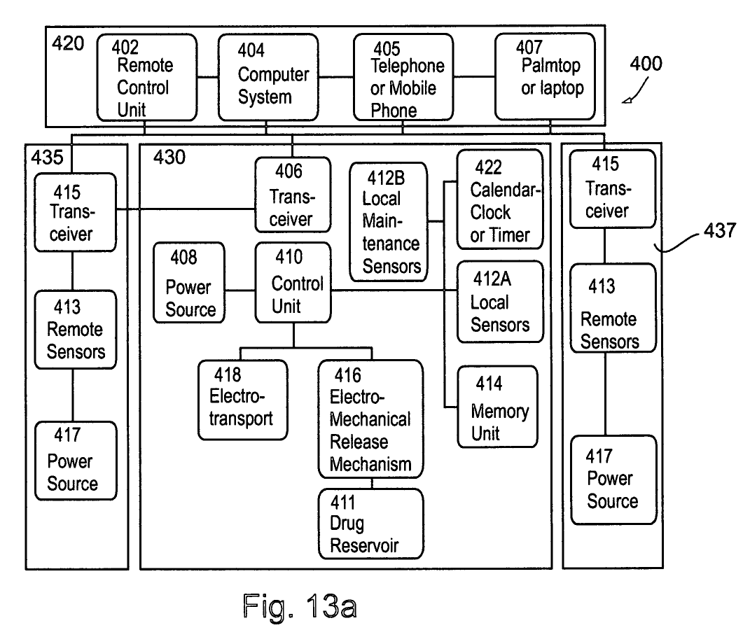

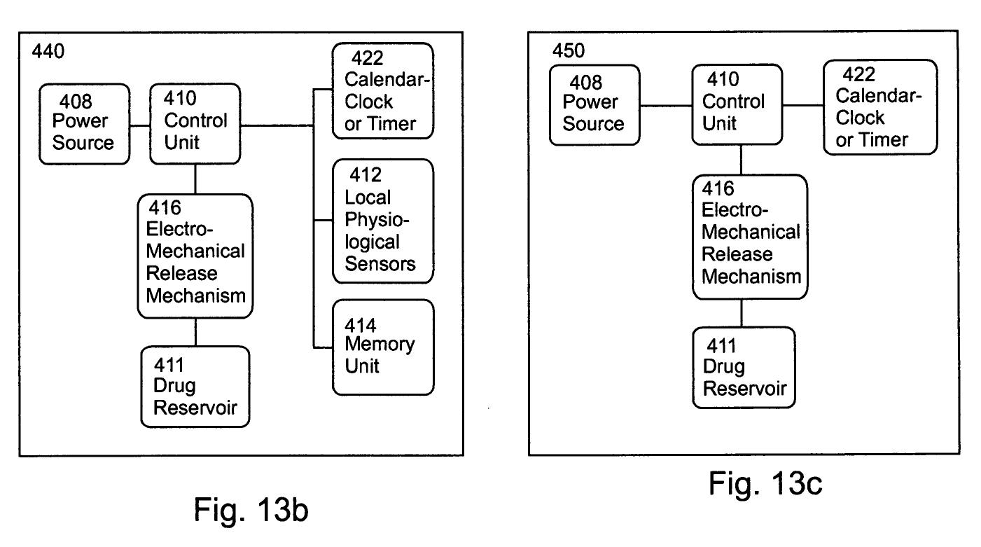

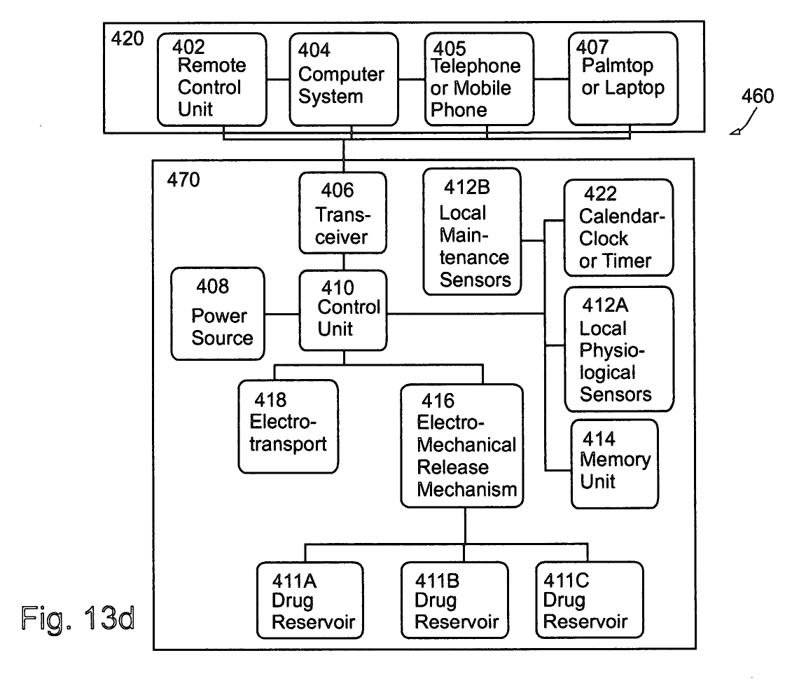

Referring further to the drawings, FIGS. 13A-13D are schematic diagrams of devices for electronic, controlled drug release, in accordance with preferred embodiments of the present invention.

As seen in FIG. 13A, a device 400 for electronic, controlled drug release may include:

i. first intracorporeal system 430, containing a drug reservoir;

ii. second intracorporeal system 435 of remote sensors;

iii. first, personal extracorporeal system 420 of remote control units; and

iv. second extracorporeal system 437, of remote sensors.

In the Figure, the intracorporeal systems are lightly shaded and the extracorporeal systems are darkly shaded.

First intracorporeal system 430 includes a drug reservoir 411, and a control unit 410 primarily for operating an electromechanical release mechanism 416, and for setting the release rate. Control unit 410 may be any one of a dedicated control circuitry 410, a processor 410, an ASIC 410, or a microcomputer 410, as known, and may further include a memory unit 414, preferably integrated with it. A power source 408 provides power to intracorporeal system 430 and a transceiver 406, operating by RF, IR or ultrasound, provides communication with personal extracorporeal system 420 of remote control units and possibly also, with second intracorporeal system 435 and second extracorporeal system 437, both of remote sensors.

First intracorporeal system 430 may further include one or several local physiological sensors 412A, one or several status sensors 412B and a timing device 422, preferably comprising a timer, a clock, and a calendar, for chronotherapy.

Control unit 410 activates electromechanical release mechanism 416, for drug release from drug reservoir 411, preferably by means of built-in intelligence and algorithms, for drug release, which may be responsive to the communicated measurements of local sensors 412 and (or) remote sensors 413, to compensate for the measurement, to correct a situation that is indicated by it, and (or) to improve the efficacy and to optimize the drug release, for an optimal closed-loop operation, or to calibrate the drug release with the measured data, in order to arrive at an optimal release schedule. Additionally or alternatively, control unit 410 may activate electro-mechanical release mechanism 416 in response to input from timing device 422, or in response to a demand from personal extracorporeal system 420. Additionally or alternatively, control unit 410 may be preprogrammed for a specific drug release schedule, which may take any one of the following forms: release at a controlled rate, delayed release, pulsatile release, and chronotherapeutic release.

Additionally, intracorporeal system 430 may further include at least one or several electrodes, coils or transducers 418 for one or several electrotransport mechanisms, sonophoresis, and (or) ablation, controlled by control unit 410, for enhanced buccal and sublingual administration.

Second intracorporeal system 435 includes remote sensors 413, a power source 417, and a transceiver 415, and may report its measurements directly to first intracorporeal system 430 or to extracorporeal system 420.

Similarly, second personal extracorporeal system 437, which is preferably attached to the skin of the person receiving the drug, includes remote sensors 413, a power source 417, and a transceiver 415, and may report its measurements directly to first intracorporeal system 430 or to extracorporeal system 420.

Personal extracorporeal system 420 may be any one of a remote-control unit 402, a computer system 404, a telephone or mobile phone 405, and (or) a palmtop or laptop 407. These may be in communication with each other, with first intracorporeal system 430, of the drug reservoir, with second intracorporeal system 435 and second personal extracorporeal system 437, both of remote sensors, and serve as intermediaries between them and monitoring center 500 (FIG. 9I).

FIG. 13B illustrates a device 440 for electronic, controlled drug release, with no remote control features. Device 440 may be preprogrammed, for a desired release schedule from drug reservoir 411. Additionally, a closed-loop operation, in which drug release is activated by physiological sensors 412 or by timing device 422 may be employed. Device 440 may further include remote sensors. A transceiver, may be added for providing communication between the remote sensors and control unit 410.

A far simpler device 450 for electronic, controlled drug release is seen in FIG. 13C, which has no remote control features, and no sensors. Device 450 preferably includes a dedicated control circuitry 452, timing device 422, power source 408 and electromechanical release mechanism 416, in addition to drug reservoir 411, containing the drug.

A device 460, which combines passive and electronic controlled release is seen in FIG. 13D. Device 460 includes two or more drug reservoirs, such as drug reservoirs 411A, 411B and 411C, each having a drug in a passive, controlled release dosage form, for example, as taught in conjunction with FIGS. 8A-8B.

In accordance with a first embodiment, the drug is to be released continuously. Thus, upon insertion into the oral cavity, electromechanical release mechanism 416 opens first drug reservoir 411A, and the drug is released to the oral cavity and tissue. When first drug reservoir 411A is depleted, electromechanical release mechanism 416 opens second drug reservoir 411B, and when that is depleted, electro-mechanical release mechanism 416 opens third drug reservoir 411C. In this manner, the interval between drug replacements can be extended considerably.

In accordance with a second embodiment, a dosage is to be released on demand. The demand may be, for example, from a remote-control unit, such as palmtop 407, for example, in response to a sudden pain. Alternatively, the demand may be responsive to a sensor reading, for example, of glucose level or of heart rate. Alternatively, the demand may be responsive to timing device 422. Each time a demand is made, electromechanical release mechanism 416 opens a drug reservoir, from among drug reservoirs 411A, 411B, and 411C, and allows the reservoir to be depleted. When all the drug reservoirs are depleted, replacement is necessary.

Device 460 of FIG. 13D may further include personal extracorporeal system 420, and possibly also extracorporeal and intracorporeal remote sensor systems, such as systems 435 and 437 of FIG. 13A.

For optimal placement and (or) anchoring of a device for controlled drug release in an oral cavity of a person, in accordance with the present invention, a dentist may examine the mouth of the person. If the patient has a dental implement, such as a crown, a prosthetic tooth crown, a bridge, dentures, braces, a night guard or a mouth guard, any one of these may be replaced with devices in accordance with the present invention. Alternatively or additionally, the patient may be in need of a dental implement, such as a crown, a prosthetic tooth crown, a bridge, dentures, braces, a night guard or a mouth guard, the needed implement may be prepared so as to include a device in accordance with the present invention. Alternatively or additionally, a wisdom tooth may be missing either because it has not yet emerged, or because it has been extracted, and that space may be used for a device in accordance with the present invention, for example, attached to a molar band, as taught in conjunction with FIG. 12D. Alternatively or additionally, a device may be mounted on a braces plate, even where braces need not be used, for dental reasons, as taught in conjunction with FIG. 12F. Alternatively or additionally, a device may be mounted on a night guard or a mouth guard, even where it need not be used for dental reasons. It will be appreciated that a combination of the above may be used.

It will be appreciated that the dosage form or electronic device for controlled drug release may be mounted on any anchor that may be secured to the oral mucosa or the jawbone. Alternatively, the dosage form or electronic device for controlled drug release may be directly implanted into a tissue without a specific anchoring element.

It will be appreciated that other known anchoring devices, for example as described in U.S. Pat. Nos. 4,175,326, 4,020,558, and 4,681,544 may be used for anchoring devices for controlled drug release, in accordance with the present invention.

Drug candidates for the present invention include antiarthritics, antibiotics, anticoagulant antagonists, antihypertensive medications, antineoplastics, and antirheumatic agents.

Additionally, blood modifiers may be used, for example, anticoagulants, antiplatelet agents, and thrombolytic agents.

Furthermore, cardiovascular agents may be used, for example, adrenergic blockers (central, peripheral and combinations), alpha/beta adrenergic blockers, angiotensin convertin enzyme inhibitors, angiotensin convertin enzyme inhibitors with calcium channel blockers, angiotensin convertin enzyme inhibitors with diuretics, angiotensin II receptor antagonists, angiotensin II receptor antagonists with diuretics, antiarrhythmics (Groups I, II, III, miscellaneous), antilipemic agents, HMG-CoA reductase inhibitors, nicotinic acid, beta adrenergic blocking agents, beta adrenergic blocking agents with diuretics, calcium channel blockers, miscellaneous cardiovascular agents, vasodilators (coronary, peripheral, pulmonary and combinations), and vasopressors.

Additionally, respiratory agents may be used, for example, bronchodilators, sympathomimetics and combinations, xanthine derivatives and combinations, miscellaneous respiratory agents, and respiratory stimulants.

Furthermore, skin and mucous membrane agents may be used, for example, antihistamines and combinations, and antineoplastics.

Additionally, viagra and similar agents may be used.

Additionally, antidepressants, and drugs for mental diseases may be used.

Furthermore, insulin and similar agents may be used.

Additionally, drugs for local therapies may be used, for example:

i. glucocorticosteroids such as betamethasone, triamcinolone, fluocinolone and similar drugs,

ii. antifungals, such as econazole, miconazole, clotrimazole, bifonazole, ketoconazole, and itraconazole;

iii. antivirals, such as acyclovir; and

iv. antibiotics, such as cefazolin, amoxycillin, vancomycin, gentamicine, and chloramphenicol.

Furthermore, drugs for systemic and chronic therapies may be used, for example:

i. antineoplastics, such as 5-fluorouracil, ftorafur, and hydroxyurea;

ii. antiepileptics, such as carbamazepine, valproate, perfenazine, phenytoine, and primidone;

iii. antiarrhythmics, such as atenolol, and timolol;

iv. antihypertensives, such as enalapril;

v. anti-HIV drugs, such as AZT;

vi. immunosuppressive agents, such as sirolimus, and tacrolimus;

vii. CNS candidates, such as galantamine;

viii. Alzheimer disease drugs, such as risperidone;

ix. drug-addiction treatment, such as buprenorphine, and naloxone;

x. chronic pain/palliative tumour therapy, such as opiate or opiate-like medication; and

xi. rheumatic pain, such as non-steroidal anti-inflammatory medication.

Additionally, drugs for diseases with a circadian pattern may be used.

Additionally, other drugs may be used.

The drugs contained in the devices in accordance with the present invention may be of large molecules, peptide drugs, or others, which might be absorbed in the general circulation directly from the oral cavity or oral tissues, without passing through the Gastrointestinal tract with all its limitations. As such, the present invention offers an alternative approach to gastro retentive systems, as well as to conventional buccal and sublingual administration and to conventional oral controlled release dosage forms.

Additionally, the drugs included in the devices may be of any type regarding its physical and chemical properties. In case of poorly soluble drugs, improved solubility approaches, such as complexation or sub-micronization (nano-systems), stabilized in any manner suitable for improved solubility, may be used.

### EXAMPLES

Reference is now made to the following examples, which together with the above description illustrate the invention in a non-limiting fashion.

### Example 1

### Passive, Controlled Drug Release

Device 140, designed as prosthetic tooth crown 160 (FIGS. 8A-8B) for passive, controlled drug release, or another device for passive, controlled drug release may include drug reservoir 156, in a dosage form of a tablet which contains cyclosporine, coated with a semi-permeable membrane that controls the drug release by osmosis. The semi-permeable is formed of hydrophobic polymers, such as cellulose acetate, or ethocel, mixed with water soluble additives, such as sugar, PEG's, and the like. Upon administration, the soluble additives dissolves and a semipermeable membrane is created. The cyclosporine is released at a rate of 0.5-2 mg per day, continuously. The tablet may be replaced about once a month. By comparison, when ingested, gastro-retention in the upper gastrointestinal tract generally does not exceed about 12 hours.

In a similar manner, levodopa may be used, in place of cyclosporine. Alternatively, growth hormones, combined with stabilizers, may be used, in place of cyclosporine.

### Example 2

### Delayed, Passive, Controlled Drug Release

Device 140, designed as prosthetic tooth crown 160 (FIGS. 8A-8B) for passive, controlled drug release, or another device for passive, controlled drug release may include several drug reservoirs 156, wherein a first reservoir includes a dosage form adapted for passive, controlled release, for example, by diffusion and erosion, and a second drug reservoir includes a dosage form which is coated by a special functional coating, designed to delay the release from the second reservoir until the dosage form of the first reservoir is depleted. In this manner, the interval between replacements may be extended.

### Example 3

### Pulsatile, Passive, Controlled Drug Release

Device 140, designed as prosthetic tooth crown 160 (FIGS. 8A-8B) for passive, controlled drug release, or another device for passive, controlled drug release may include a drug reservoir 156, which includes a dosage form having a multi-layer coating, designed for pulsatile passive controlled release, which may be synchronized, for example, with circadian cycles, for a desired chronotherapy.

### Example 4

### Passive, Controlled Drug Release

Prosthetic tooth crown 160 (FIGS. 8A-8B) for passive, controlled drug release, or another device for passive, controlled drug release may include drug reservoir 156 of the anti HIV drug AZT, incorporated into pellets or minitabs. The release mechanism is diffusion or erosion. The dosage form is replaced once a week.

### Example 5

### Electronic and Passive Controlled Drug Release

Electronic, controlled drug release device 460 (FIG. 13D) may include two or more drug reservoirs, such as 411A, 411B, and 411C of the anti HIV drug AZT, incorporated into pellets or minitabs, of a passive, controlled release dosage form, which may last about a week. Upon insertion, electromechanical release mechanism 416 opens first drug reservoir 411A, and controlled release by diffusion takes place. When the first reservoir 4111A is depleted, status sensor 412B informs control unit 410, and control unit 410 instructs electromechanical release mechanism 416 to open second drug reservoir 411B. About a week later, second reservoir is depleted, and third drug reservoir 411C is opened. In this manner, replacement intervals are extended from one week, as in Example 2, to several weeks, depending on the number of drug reservoirs.

### Example 6

### Chronotherapy Drug Release for Cancer

According to Stehlin [Stehlin I., “A Time to Heal: Chronotherapy Tunes In to Body's Rhythms,” US Food and Drug Administration, http:/www.fda.gov/fdac/features/1997/397_chrono.html], chronotherapy may be useful in the treatment of cancer. Animal studies suggest that chemotherapy may be more effective and less toxic if cancer drugs are administered at carefully selected times. It appears that there may be different chronobiological cycles for normal cells and tumor cells. Thus, if administration of cancer drugs is timed with the chronobiological cycles of tumor cells, it will be more effective against the cancer and less toxic to normal tissues. Thus, any one of device 400, 440, 450, or 460, for electronic, controlled drug release (FIGS. 13A-13D), may be preprogrammed for clock operated drug release, for example, of chemotherapy, for chronotherapy.

By using any one of device 400, 440, 450, or 460, for electronic, controlled drug release (FIGS. 13A-13D), drug released may be synchronized with either predetermined patterns or real-time measurements of physiological parameters. Thus the cancer patient receives the cancer drugs in an effective way, with minimal side effects and waste.

### Example 7

### Chronotherapy and Remote Control Drug Release for Arthritis

According to Stehlin [Stehlin I., “A Time to Heal: Chronotherapy Tunes In to Body's Rhythms,” US Food and Drug Administration, http://www.fda.gov/fdac/features/1997/397_chrono.html], chronotherapy may be useful in the treatment of arthritis. People with osteoarthritis tend to have less pain in the morning and more at night; while those with rheumatoid arthritis, have pain that usually peaks in the morning and decreases throughout the day. Chronotherapy for all forms of arthritis using NSAIDs such as ibuprofen may be timed to ensure that the highest blood levels of the drug coincide with peak pain. Devices 400 or 460, for electronic, controlled drug release (FIGS. 13A and 13D), may be preprogrammed for clock-operated drug release, synchronized to the circadian rhythm of the disease, based on the patient's history, for chronotherapy.

Chronotherapy may be supplemented by remote control operation, from personal extracorporeal system 420, preferably by the patient, for example, from remote-control unit 402, palmtop 407, or another remote-control unit, when a patient feels pain.

### Example 8

### Chronotherapy, Remote Control and Sensor-Activated Drug Release for Diabetes

Glucose levels vary throughout the day, to some extent in a cyclic manner. Additionally, there is a rise in glucose level shortly after eating. Devices 400 or 460, for electronic, controlled drug release (FIGS. 13A and 13D), may be pre-programmed for clock operated drug release, synchronized to the circadian rhythm of the glucose, for chronotherapy. Preferably, the synchronization is based on the patient's history of glucose level cyclic variations.

Chronotherapy may be supplemented by remote control operation, from personal extracorporeal system 420, preferably by the patient, for example, from remote-control unit 402, palmtop 407, or another remote-control unit, when a patient is about to eat, since he knows that glucose levels will rise then.

Additionally, remote control operation may be performed, responsive to a report from one or several sensors 413, that glucose levels in the blood or in the interstitial fluid have risen. The remote control operation, from personal extracorporeal system 420, may be by the patient, for example, from remote-control unit 402 or palmtop unit 407, upon the patient's seeing the glucose level measurement on display. Additionally or alternatively the patient may forward the measurement to monitoring center 500 (FIG. 9I), for example, via remote-control unit 402 or palmtop unit 407, or another remote-control unit, for the monitoring center's decision, for example of computer 502, on a drug release schedule.

Alternatively, a closed-loop operation, may take place without the patient's intervention, when a glucose sensor 413 reports a measurement that leads the built-in intelligence and algorithms of the device to determine that the value is too high. The determination and demand for drug release may be made directly by control unit 410, for example, of intracorporeal system 430 of device 400, based on its built-in intelligence and algorithms, for drug release responsive to the communicated measurements. Alternatively, the determination and demand for drug release may come from computer system 502 (FIG. 9I) of monitoring center 500, wherein the sensor measurements are forwarded to monitoring center 500, for example, by remote-control unit 402 or palmtop unit 407, or another remote-control unit, and these also receive the instructions from monitoring center 500 and pass it on to control unit 410 of intracorporeal system 430. In this manner, drug release may take place by remote control even without the patient's being aware of it, and drug release may accurately match the patient's needs.

### Example 9

### Chronotherapy, Remote Control and Sensor-Activated Drug Release for Asthma

According to Stehlin [Stehlin I., “A Time to Heal: Chronotherapy Tunes In to Body's Rhythms,” US Food and Drug Administration, http://www.fda.gov/fdac/features/1997/397_chrono.html], chronotherapy may be useful in the treatment of asthma, since asthmatic patients tend to have attacks during the early hours of the morning, for example, between 3 and 5 AM. Devices 400 or 460, for electronic, controlled drug release (FIGS. 13A and 13D), may be preprogrammed for clock operated drug release, synchronized to the circadian rhythm of the disease, for chronotherapy, which at times may be further supplemented by remote control operation. The released drug may be, for example, the bronchodilator, Uniphyl. The dosage form may be a tablet, minitab, and the like, but may include some formulative modifications. Replacement may take place about once a week, as with other dosage forms.

Synchronization may be performed on a case by case basis, by preprogramming the device, based on the patient history of the disease.

Additionally or alternatively, the drug release rate may be increased a little before the expected time for the attack.

Chronotherapy may be supplemented by remote control operation, from personal extracorporeal system 420, preferably by the patient, for example, from remote-control unit 402 or palmtop unit 407, or another remote-control unit, when a patient feels the onset of an attack.

Additionally or alternatively, chronotherapy may be supplemented by a closed-loop operation, which may be without the patient's intervention, when any of the physiological sensors 413, for example, heart-rate sensor 413, reports a measurement that leads the built-in intelligence and algorithms of the device to determine the onset of an attack. The determination and demand for drug release may be made directly by control unit 410, for example, of intracorporeal system 430 of device 400, based on its built-in intelligence and algorithms, for drug release responsive to the communicated measurements. Alternatively, the determination and demand for drug release may come from personal extracorporeal system 420, for example, from computer system 404. Alternatively, the determination and demand for drug release may come from monitoring center 500 (FIG. 9I), wherein the sensor measurements are forwarded to the monitoring center, for example, by remote-control unit 402 or palmtop unit 407, or another remote-control unit, and these also receive the instructions from monitoring center 500 and pass it on to control unit 410 of intracorporeal system 430. In this manner, drug release may take place by remote control even as the patient sleeps.

### Example 10

### Sensor-Activated Drug Release for Snoring and Other Sleeping Disorders

For sleeping disorder, a closed loop operation is probably most suitable and device 440 of FIG. 13B may be used. Sensors 412 may be piezo-electric transducers, which sense sound, such as snoring, or heartbeat. The determination and demand for drug release may be made directly by control unit 410, based on its built-in intelligence and algorithms, for drug release responsive to the communicated measurements. For snoring the communicated measurement may be the sound of snoring. For insomnia, the communicated measurement may be the rate of hearbeat, indicating whether the patient is asleep or awake.

### Example 11

### Remote Control Drug Release for Mental Diseases

Devices 400 or 460, for electronic, controlled drug release (FIGS. 13A and 13D) may be used by patients suffering from mental conditions such as depression or hypertension. When the situation deteriorates, either the patient, or a caretaker such as a parent may initiate drug release, for example, via remote-control unit 202, palmtop 407, or another remote-control unit.

For geriatric patients, suffering from senility or Alzheimer, sensors 412 or 413 may further include a global positioning device, and these may also be mounted on remote-control unit 202, and (or) palmtop 407, or another remote-control unit, for reporting both the location of the patient and of the remote-control unit to the monitoring center.

### Example 12

### Sexual Dysfunction

Devices 400 or 460 for electronic, controlled drug release, may be used for sexual dysfunction, wherein when wishing to be aroused, a person uses remote-control unit 402 or palmtop 407, or another remote-control unit, for the release of an arousing drug, such as Viagra.

### Example 13

### Narcotic Rehabilitation

When using device 400 or 460, having status sensors, to determine and report the amount of drug remaining in the drug reservoir, for example, on display on remote-control unit 402 or palmtop 407, or another remote-control unit, the user may observe and actively participate in the drug usage rate. Thus, the user may set up goals for himself, to reduce the drug release rate, at small increments, until rehabilitation.

### Example 14

### Narrow-Therapeutic-Index Drugs

Devices 400 or 460 for electronic, controlled drug release, which include remote sensors 413 for drug concentration levels in the blood or in the interstitial fluid may be used for drugs of narrow therapeutic indexes, wherein the drug concentration in the blood or interstitial fluid is monitored, preferably continuously, and drug release is responsive to the monitoring.

### Example 15

### Economic Use of Drugs

Many of today's drugs are very expensive. Yet when orally administered, only a portion of the dosage form is utilized while the rest may reach the colon and is eliminated by the body. When implanted in the mouth cavity and released in a control manner, waste of the drug is greatly reduced.

Additionally, When using device 400 or 460, having status sensors, to determine and report the amount of drug remaining in the drug reservoir, for example, by display on remote-control unit 402 or palmtop 407, or another remote-control unit, the drug is only replaced when needed, and unused drug is not discarded.

### Example 16

### Personalized Drug Administration Based on DNA Analysis

Drug release schedule may be based on DNA reconstruction and analysis, to match each patient's DNA. DNA parameters may be processed prior to the drug administration, or during it, to define the best drug administration policy for a particular patient. A-DNA dependent release schedule may occur, for example, in consequence to a determination that the patient's DNA includes a gene that makes that patient more susceptible to certain diseases, such as, breast cancer, or heart attacks.

### Example 17

### Personalized Drug Administration Based on Physical Parameters and Personal History

Drug release schedule may be based on physical-parameters and personal-history analyses, so as to be tuned to a specific patient. Physical-parameters and personal-history analyses may include patient's weight, height, age, gender, physiological history, medical status, other medication administrated simultaneously, blood pressure, blood analysis and the like. These parameters may be processed prior to the drug administration, or during it, to define the drug administration policy that will achieve best results for a particular patient.

It is expected that during the life of this patent many relevant oral devices and methods controlled drug release will be developed and the scope of the term substances, devices, and methods for photo-sterilization is intended to include all such new technologies a priori.

As used herein the term “about” refers to ±30%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for controlled drug release, comprising:
a reservoir containing a drug; and
an electronic drug release mechanism, for providing said controlled drug release,
the device being adapted for insertion to an oral cavity of a subject.

2. The device of claim 1, wherein said electronic drug release mechanism further includes:
a control unit, for controlling said controlled release;
an electro-mechanical release mechanism, which opens to allow the release of said drug, responsive to commands from said control unit; and
a power source, for powering said control unit and electromechanical release mechanism.

3. The device of claim 2, wherein said control unit is selected from the group consisting of a dedicated electronic circuitry, a processor, an ASIC, and a microcomputer.

4. The device of claim 1, wherein said device for controlled drug release further includes a timing device, selected from the group consisting of a timer, a clock, a calendar, and a combination thereof.

5. The device of claim 1, and further including at least one local sensor, integrated with said device.

6. The device of claim 5, and further including at least two local sensors, integrated with said device.

7. The device of claim 5, wherein said at least one local sensor is a physiological sensor, for drug release responsive to measurements of said sensor.

8. The device of claim 7, wherein said local physiological sensor is selected from the group consisting of a sensor for drug concentration in the saliva, a sensor for glucose concentration in the saliva, a sensor for a metabolite concentration in the saliva, a sensor for an electrolyte concentration in the saliva, a sensor for the pH level in the saliva, a sensor for the temperature in the oral cavity, a heartbeat sensor, a heart rate sensor, and a snoring sensor.

9. The device of claim 5, wherein said at least one local sensor is a status sensor, for ensuring that the device is in proper operating condition.

10. The device of claim 9, wherein said local status sensor is selected from the group consisting of a sensor for remaining drug in the drug reservoir, a sensor for drug flow rate, a sensor for power source condition, and a sensor for short-circuit detection.

11. The device of claim 1, and further including at least one communication component, selected from the group consisting of a receiver, a transmitter, and a transceiver.

12. The device of claim 11, wherein said communication component provides communication with a personal extracorporeal system.

13. The device of claim 12, wherein said personal extracorporeal system is selected from the group consisting of a remote control unit, a computer system, a telephone, a mobile phone, a palmtop, a PDA, a laptop, and a combination thereof.

14. The device of claim 13, wherein said personal extracorporeal system is adapted to provide communication between said device and a monitoring center.

15. The device of claim 11, wherein said communication component provides communication with at least one remote sensor.

16. The device of claim 15, wherein said remote sensor is selected from the group consisting of a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, a temperature sensor, a heartbeat sensor, a heart rate sensor, and a snoring sensor.

17. The device of claim 1, wherein said device further includes at least one drug-transfer component for increased drug transfer through a biological barrier, by a process selected from the group consisting of iontophoresis, electroosmosis, electrophoresis, electroporation, sonophoresis, and ablation.

18. The device of claim 1, wherein said drug release mechanism provides said controlled drug release in a manner selected from the group consisting of release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system.

19. The device of claim 1, and further including at least two drug reservoirs.

20. The device of claim 1, wherein said drug is in nano-size particles.

21. The device of claim 1, wherein said device is mounted on a dental implement, designed for the oral cavity of the subject.

22. The device of claim 21, wherein said dental implement is selected from the group consisitng of a prosthetic tooth crown, a dental bridge, a dental three-unit bridge, dental implant, partial dentures, full dentures, braces, a molar band, a night guard, and a mouth guard.

23. The device of claim 1, wherein said device is mounted on an anchor that may be secured to the oral mucosa or the jawbone.

24. The device of claim 1, wherein said device is anchor-free, and is directly implanted into a tissue.

25. The device of claim 1, wherein said device is adapted to be removably inserted to the oral cavity of the subject.

26. The device of claim 1, wherein said device is adapted to be permanetly inserted to the oral cavity of the subject.

27. The device of claim 1, wherein said device is adapted to be permanetly inserted to the oral cavity of the subject, and said device further includes a removable component, which houses at least one of said drug reservoir and said power source.

28. A method of controlled drug release, comprising:
providing a device for controlled drug release, which comprises a reservoir containing a drug and an electronic drug release mechanism for controllably releasing said drug; and
inserting said device to an oral cavity of a subject.

29. The method of claim 28, wherein said electronic drug release mechanism further includes:
a control unit, for controlling said controlled release;
an electromechanical release mechanism, which opens to allow the release of said drug, responsive to commands from said control unit; and
a power source, for powering said control unit and electromechanical release mechanism.

30. The method of claim 29, wherein said control unit is selected from the group consisting of a dedicated electronic circuitry, a processor, an ASIC, and a microcomputer.

31. The method of claim 28, wherein said device for controlled drug release further includes a timing device, selected from the group consisting of a timer, a clock, a calendar, and a combination thereof.

32. The method of claim 28, and further including at least one local sensor, integrated with said device.

33. The method of claim 32, and further including at least two local sensors, integrated with said device.

34. The method of claim 32, wherein said at least one local sensor is a physiological sensor, for drug release responsive to measurements of said sensor.

35. The method of claim 34, wherein said local physiological sensor is selected from the group consisting of a sensor for drug concentration in the saliva, a sensor for glucose concentration in the saliva, a sensor for a metabolite concentration in the saliva, a sensor for an electrolyte concentration in the saliva, a sensor for the pH level in the saliva, a sensor for the temperature in the oral cavity, a heartbeat sensor, a heart rate sensor, and a snoring sensor.

36. The method of claim 32, wherein said at least one local sensor is a status sensor, for ensuring that the device is in proper operating condition.

37. The method of claim 36, wherein said local status sensor is selected from the group consisting of a sensor for remaining drug in the drug reservoir, a sensor for drug flow rate, a sensor for power source condition, and a sensor for short-circuit detection.

38. The method of claim 28, and further including at least one communication component, selected from the group consisting of a receiver, a transmitter, and a transceiver.

39. The method of claim 38, wherein said communication component provides communication with a personal extracorporeal system.

40. The method of claim 39, wherein said personal extracorporeal system is selected from the group consisting of a remote control unit, a computer system, a telephone, a mobile phone, a palmtop, a PDA, a laptop, and a combination thereof.

41. The method of claim 40, wherein said personal extracorporeal system is adapted to provide communication between said device and a monitoring center.

42. The method of claim 38, wherein said communication component provides communication with at least one remote sensor.

43. The method of claim 42, wherein said remote sensor is selected from the group consisting of a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, temperature sensor, a heartbeat sensor, a heart rate sensor, and a snoring sensor.

44. The method of claim 28, wherein said device further includes at least one drug-transfer component for increased drug transfer through a biological barrier, by a process selected from the group consisting of iontophoresis, electroosmosis, electrophoresis, electroporation, sonophoresis, and ablation.

45. The method of claim 28, wherein said drug release mechanism provides said controlled drug release in a manner selected from the group consisting of release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system.

46. The method of claim 28, and further including at least two drug reservoirs.

47. The method of claim 28, wherein said drug is in nano-size particles.

48. The method of claim 28, wherein said device is mounted on a dental implement, designed for the oral cavity of the subject.

49. The method of claim 48, wherein said dental implement is selected from the group consisitng of a prosthetic tooth crown, a dental bridge, a dental three-unit bridge, dental implant, partial dentures, full dentures, braces, a molar band, a night guard, and a mouth guard.

50. The method of claim 28, wherein said device is mounted on an anchor that may be secured to the oral mucosa or the jawbone.

51. The method of claim 28, wherein said device is anchor-free, and is directly implanted into a tissue.

52. The method of claim 28, wherein said device is adapted to be removably inserted to the oral cavity of the subject.

53. The method of claim 28, wherein said device is adapted to be permanently inserted to the oral cavity of the subject.

54. The method of claim 28, wherein said device is adapted to be permanetly inserted to the oral cavity of the subject, and said device further includes a removable component, which houses at least one of said drug reservoir and said power source.

55. A device for controlled drug release, comprising:
a reservoir containing a drug; and
a dental implement, designed to be inserted to the oral cavity of a subject, and adapted for supporting said drug reservoir.

56. The device of claim 55, wherein said dental implement is selected from the group consisitng of a prosthetic tooth crown, a dental bridge, a dental three-unit bridge, dental implant, partial dentures, full dentures, braces, a molar band, a night guard, and a mouth guard.

57. The device of claim 55, wherein said dental implement is designed to be removably inserted to the oral cavity of a subject.

58. The device of claim 55, wherein said dental implement is designed to be permanently inserted to the oral cavity of a subject.

59. The device of claim 55, wherein said dental implement is designed to be permanetly inserted to the oral cavity of the subject, and said dental implement further includes a removable component, which houses at least one of said drug reservoir and said power source.

60. The device of claim 55, wherein said drug reservoir contains a drug is a dosage form for passive, controlled drug release.

61. The device of claim 55, wherein said drug reservoir contains a drug is a dosage form of nano-size particles.

62. The device of claim 55, and further including an electronic drug release mechanism.

63. The device of claim 62, wherein said drug is in a controlled release dosage form for a combination of electronic and passive controlled release.

64. The device of claim 63, and further including at least two drug reservoirs.

65. The device of claim 55, wherein said electronic drug release mechanism further includes:
a control unit, for controlling said controlled release;
an electromechanical release mechanism, which opens to allow the release of said drug, responsive to commands from said control unit; and
a power source, for powering said control unit and electromechanical release mechanism.

66. The device of claim 65, wherein said control unit is selected from the group consisting of a dedicated electronic circuitry, a processor, an ASIC, and a microcomputer.

67. The device of claim 55, wherein said device for controlled drug release further includes a timing device, selected from the group consisting of a timer, a clock, a calendar, and a combination thereof.

68. The device of claim 55, and further including at least one local sensor, integrated with said device.

69. The device of claim 68, wherein said at least one local sensor is a physiological sensor, for drug release responsive to measurements of said sensor.

70. The device of claim 69, wherein said local physiological sensor is selected from the group consisting of a sensor for drug concentration in the saliva, a sensor for glucose concentration in the saliva, a sensor for a metabolite concentration in the saliva, a sensor for an electrolyte concentration in the saliva, a sensor for the pH level in the saliva, a sensor for the temperature in the oral cavity, a heartbeat sensor, a heart rate sensor, and a snoring sensor.

71. The device of claim 68, wherein said at least one local sensor is a status sensor, for ensuring that the device is in proper operating condition.

72. The device of claim 71, wherein said local status sensor is selected from the group consisting of a sensor for remaining drug in the drug reservoir, a sensor for drug flow rate, a sensor for power source condition, and a sensor for short-circuit detection.

73. The device of claim 55, and further including at least two local sensors, integrated with said device.

74. The device of claim 55, and further including at least one communication component, selected from the group consisting of a receiver, a transmitter, and a transceiver.

75. The device of claim 74, wherein said communication component provides communication with a personal extracorporeal system.

76. The device of claim 75, wherein said local extracorporeal system is selected from the group consisting of a remote control unit, a computer system, a telephone, a mobile phone, a palmtop, a PDA, a laptop, and a combination thereof.

77. The device of claim 76, wherein said personal extracorporeal system is adapted to provide communication between said device and a monitoring center.

78. The device of claim 74, wherein said communication component provides communication with at least one remote sensor.

79. The device of claim 78, wherein said remote sensor is selected from the group consisting of a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, a temperature sensor, a heartbeat sensor, a heart rate sensor, and a snoring sensor.

80. The device of claim 55, wherein said device further includes at least one drug-transfer component for increased drug transfer through a biological barrier, by a process selected from the group consisting of iontophoresis, electroosmosis, electrophoresis, electroporation, sonophoresis, and ablation.

81. The device of claim 55, wherein said drug release mechanism provides said controlled drug release in a manner selected from the group consisting of release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system.

82. The device of claim 55, wherein said drug is in nano-size particles.

83. A method of controlled drug release, comprising:
providing a device for controlled drug release, which comprises a reservoir containing a drug; and
supporting said device in an oral cavity of a subject, on a dental implement, designed for insertion to the oral cavity of a subject and for supporting the device.

84. The method of claim 83, wherein said dental implement is selected from the group consisitng of a prosthetic tooth crown, a dental bridge, a dental three-unit bridge, dental implant, partial dentures, full dentures, braces, a molar band, a night guard, and a mouth guard.

85. The method of claim 83, wherein said dental implement is designed to be removably inserted to the oral cavity of a subject.

86. The method of claim 83, wherein said dental implement is designed to be permanently inserted to the oral cavity of a subject.

87. The method of claim 83, wherein said dental implement is designed to be permanetly inserted to the oral cavity of the subject, and said dental implement further includes a removable component, which houses at least one of said drug reservoir and said power source.

88. The method of claim 83, wherein said drug reservoir contains a drug is a dosage form for passive, controlled drug release.

89. The method of claim 83, wherein said drug reservoir contains a drug is a dosage form of nano-size particles.

90. The method of claim 83, and further including an electronic drug release mechanism.

91. The method of claim 90, wherein said drug is in a controlled release dosage form for a combination of electronic and passive controlled release.

92. The method of claim 91, and further including at least two drug reservoirs.

93. The method of claim 83, wherein said electronic drug release mechanism further includes:
a control unit, for controlling said controlled release;
an electro-mechanical release mechanism, which opens to allow the release of said drug, responsive to commands from said control unit; and
a power source, for powering said control unit and electromechanical release mechanism.

94. The method of claim 93, wherein said control unit is selected from the group consisting of a dedicated electronic circuitry, a processor, an ASIC, and a microcomputer.

95. The method of claim 83, wherein said device for controlled drug release further includes a timing device, selected from the group consisting of a timer, a clock, a calendar, and a combination thereof.

96. The method of claim 83, and further including at least one local sensor, integrated with said device.

97. The method of claim 96, wherein said at least one local sensor is a physiological sensor, for drug release responsive to measurements of said sensor.

98. The method of claim 97, wherein said local physiological sensor is selected from the group consisting of a sensor for drug concentration in the saliva, a sensor for glucose concentration in the saliva, a sensor for a metabolite concentration in the saliva, a sensor for an electrolyte concentration in the saliva, a sensor for the pH level in the saliva, a sensor for the temperature in the oral cavity, a heartbeat sensor, a heart rate sensor, and a snoring sensor.

99. The method of claim 96, wherein said at least one local sensor is a status sensor, for ensuring that the device is in proper operating condition.

100. The method of claim 99, wherein said local status sensor is selected from the group consisting of a sensor for remaining drug in the drug reservoir, a sensor for drug flow rate, a sensor for power source condition, and a sensor for short-circuit detection.

101. The method of claim 83, and further including at least two local sensors, integrated with said device.

102. The method of claim 83, and further including at least one communication component, selected from the group consisting of a receiver, a transmitter, and a transceiver.

103. The method of claim 102, wherein said communication component provides communication with a personal extracorporeal system.

104. The method of claim 103, wherein said local extracorporeal system is selected from the group consisting of a remote control unit, a computer system, a telephone, a mobile phone, a palmtop, a laptop, and a combination thereof.

105. The method of claim 104, wherein said personal extracorporeal system is adapted to provide communication between said device and a monitoring center.

106. The method of claim 102, wherein said communication component provides communication with at least one remote sensor.

107. The method of claim 106, wherein said remote sensor is selected from the group consisting of a sensor for drug concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for drug concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for drug concentration in the sweat, a temperature sensor, a heartbeat sensor, a heart rate sensor, and a snoring sensor.

108. The method of claim 83, wherein said device further includes at least one drug-transfer component for increased drug transfer through a biological barrier, by a process selected from the group consisting of iontophoresis, electroosmosis, electrophoresis, electroporation, sonophoresis, and ablation.

109. The method of claim 83, wherein said drug release mechanism provides said controlled drug release in a manner selected from the group consisting of release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system.

110. The method of claim 83, wherein said drug is in nano-size particles.
